(19) United States
(12) Patent Application Publication
(10) Pub. No.: US 2022/0160890 A1
(43) Pub. Date: May 26, 2022

(54) SURVIVAL-TARGETING CHIMERIC (SURTAC) MOLECULES

(71) Applicant: Locki Therapeutics Limited, London (GB)

(72) Inventor: David William SHEPPARD, Great Saxham (GB)

(73) Assignee: Locki Therapeutics Limited, London (GB)

(21) Appl. No.: 17/426,141

(22) PCT Filed: Feb. 19, 2020
(86) PCT No.: PCT/EP2020/054327

§ 371 Date: Jul. 28, 2021

Related U.S. Application Data
(60) Provisional application No. 62/808,305, filed on Feb. 21, 2019.

(51) Int. Cl. A61K 47/68; A61K 47/64; A61K 47/55; A61P 35/00

(52) U.S. Cl. CPC A61K 47/6811; A61P 35/00; A61K 47/55; A61K 47/64

(57) ABSTRACT

Survival-targeting chimeric (SURTAC) molecules are provided herein, as well as methods for their use in removing ubiquitin molecules from ubiquitinylated proteins. In one embodiment, the SURTAC molecule comprises a first binding domain, a second binding domain, and a linker domain, wherein the first binding domain is configured to bind to an ubiquitinylated protein; the second binding domain is configured to bind to an ubiquitin protease that cleaves one or more ubiquitin from the ubiquitinylated protein bound to the first binding domain, and the linker domain is configured to link the first binding domain to the second binding domain.

### FIELD OF THE INVENTION

The present disclosure is generally related to the field of bi-functional molecules. In one embodiment, provided herein are survival-targeting chimeric (SURTAC) molecules designed to decrease cellular degradation of ubiquitinylated proteins.

### BACKGROUND OF THE INVENTION

The concentration of any protein within a living cell is determined by the balance of protein synthesis and protein degradation. Regulated protein degradation is key in precisely controlling individual protein level within cells.

Proteasomes are protein complexes which degrade proteins by proteolysis, a chemical reaction that breaks peptide bonds. Enzymes that help such reactions are called proteases. The proteasomes form a pivotal component for the Ubiquitin-Proteasome System (UPS). Protein ubiquitination and subsequent proteolysis and degradation by the proteasome are important mechanisms in the regulation of the cell cycle, cell growth and differentiation, gene transcription, signal transduction and apoptosis.

As part of the UPS, the proteasome plays an essential role in malignant transformation. UPS proteolysis plays a major role in responses of cancer cells to stimulatory signals that are critical for the development of cancer. Accordingly, protein levels of transcription factors, such as p53, c-Jun, c-Fos, NF-κB, c-Myc, HIF-1α, MATα2, STAT3, sterol-regulated element-binding proteins and androgen receptors are all controlled by degradation by the UPS. Moreover, the UPS regulates the degradation of tumor suppressor gene products such as adenomatous polyposis coli (APC) in colorectal cancer, retinoblastoma (Rb). and von Hippel-Lindau tumor suppressor (VHL).

By controlling the levels of key regulatory proteins, the UPS contributes to nearly every aspect of cellular function. The UPS also functions in protein quality control, rapidly identifying and destroying misfolded proteins. Therefore, dysregulation of protein degradation pathways is critical in many human diseases.

Proteins essential for all cells may be called housekeeping proteins, suggesting that their expression is crucial for the maintenance of basic cellular function. Housekeeping genes encoding these proteins are typically constitutive genes that are required for the maintenance of basal cellular functions regardless of the housekeeping protein's specific role in the tissue or organism. A transcriptomics analysis of samples representing all major organs and tissues in the human body identified thousands of protein-coding genes detected in all analyzed tissues. Housekeeping proteins are involved in key cellular functions, such as gene expression machinery, cellular metabolism, and structural cellular proteins. In view of their crucial role in maintaining cellular homeostasis, it is clear that any deviation from normal activity of any housekeeping protein will have acute and wide-spread implication on cellular function, which may result in cellular pathogenicity and morbidity.

Exemplary housekeeping proteins include proteins of the HSP family, which are heat-shock proteins; proteins of the ATF family, which act as transcription factors; proteins of the EIF family, which act as translation factors; proteins of the EIF family, which act as translation factors; proteins of the RPL family, which are ribosomal proteins; proteins of the ARHG family, which are proteins involved in cell-cycle; and proteins of the PSMA family, which are proteins of the proteasome.

Heat shock proteins (HSP) are a family of proteins that are produced by cells in response to exposure to stressful conditions. This makes the HSP proteins “housekeeping proteins” as they are (a) activated after an insult (i.e. heat shock) to a cell, and (b) operate to return the cell to homeostasis. HSPs are activated in relation to heat shock, cold shock, exposure to UV, during wound healing and tissue remodeling. The most studied HSPs are Hsp60, Hsp70 and Hsp90.

The provision of a technology, e.g. compounds and methods, to specifically protect a protein of interest, such as a housekeeping protein, out of all the proteins in the cell's proteomic milieu from UPS-related degradation would be highly beneficial. Such technology can be used e.g. in a wide variety of therapeutic applications where over—or otherwise unwanted—UPS-related protein degradation is part of the etiology of human diseases, e.g. cancer or Cystic Fibrosis (CF). Hanna and coworkers have recently reviewed the connection between protein degradation and the pathologic basis of human diseases (The American Journal of Pathology, Vol. 189, No. 1, January 2019).

Thus, there is a need to develop compounds, which are designed to prevent UPS-related degradation of protein of interest, for therapy of various human diseases.

### SUMMARY OF THE INVENTION

In one embodiment, the present disclosure provides a survival-targeting chimeric (SURTAC) molecule comprising a first binding domain, a second binding domain, and a linker domain, wherein the first binding domain is configured to bind to an ubiquitinylated protein; the second binding domain is configured to bind to an ubiquitin protease that cleaves one or more ubiquitin molecules from the ubiquitinylated protein bound to the first binding domain, and the linker domain is configured to link the first binding domain to the second binding domain.

In one embodiment, the first binding domain comprises a peptide or a small molecule. In one embodiment, the first binding domain is configured to directly bind to the ubiquitinylated protein, for example, the first binding domain comprises an antibody or an antigen-binding fragment thereof that binds to the ubiquitinylated protein. In another embodiment, the first binding domain comprises a ligand that binds to the ubiquitinylated protein. In another embodiment, the first binding domain binds an intermediate molecule that binds to the ubiquitinylated protein.

In one embodiment, the ubiquitinylated protein bound by the first binding domain interacts with ubiquitin protease USP5. In another embodiment, the ubiquitinylated protein interacts with ubiquitin protease USP7. In another embodiment, the ubiquitinylated protein interacts with ubiquitin protease USP10. In another embodiment, the ubiquitinylated protein is a non-natural target of ubiquitin protease, for example, but not limited to, the ubiquitinylated protein is not known to be a substrate for the DUB.

In one embodiment, the second binding domain of the chimeric molecule disclosed herein may comprise a peptide or a small molecule. In one embodiment, the second binding domain is configured to directly bind to the ubiquitin protease, for example, the second binding domain comprises an antibody or an antigen-binding fragment thereof that binds to the ubiquitin protease. In another embodiment, the second binding domain comprises a ligand that binds to the ubiquitin protease. In another embodiment, the second binding domain comprises an aptamer that binds to the ubiquitin protease. In another embodiment, the second binding domain binds an intermediate molecule that binds to the ubiquitin protease.

In one embodiment, the ubiquitin protease may comprise an ubiquitin-specific proteases (DUSP) domain, an ubiquitin-like (UBL) domain, a meprin and TRAF homology (MATH) domain, a zinc-finger ubiquitin-specific protease (ZnF-UBP) domain, a zinc-finger myeloid, nervy and DEAF1 (ZnF-MYND) domain, an ubiquitin-associated (UBA) domain, a CHORD-SGT1 (CS) domain, a microtubule-interacting and trafficking (MIT) domain, a rhodenase-like domain, a TBC/RABGAP domain, a B-box domain, or any combination thereof.

In one embodiment, the ubiquitin protease is from the ubiquitin specific proteases (USP) family, the ovarian tumor proteases (OUT) family, the ubiquitin C-terminal hydrolases (UCH) family, the Josephin domain family (Josephin), the motif interacting with ubiquitin-containing novel deubiquitinase family (MINDY), or the JAB1/MPN/Mov34 metalloenzyme domain family (JAMM). For example, the ubiquitin protease can be USP5, USP7, or USP10.

In one embodiment, the linker domain of the chimeric molecule disclosed herein may comprise a peptide or a small molecule. In one embodiment, the linker domain comprises a flexible linker or a rigid linker. The linker domain may covalently link the first binding domain to the second binding domain. In another embodiment, the linker domain non-covalently links the first binding domain to the second binding domain.

### BRIEF DESCRIPTION OF THE DRAWINGS

The subject matter disclosed herein is particularly pointed out and distinctly claimed in the concluding portion of the specification. The chimeric molecules provided herein, however, both as to organization and method of operation, together with objects, features, and advantages thereof, may best be understood by reference to the following detailed description when read with the accompanying drawings in which:

Abbreviations used throughout the Figures include: ubiquitin: Ub; a deubiquitinating enzyme: a DUB; a DUB engagement motif, which may bind to a deubiquitinating enzyme (DUB): DEM (a second binding domain); a linker: LINK; a target polypeptide, which may be a ubiquitinylated (Ub) protein of interest: a TAR; and a TAR engagement motif, which may bind to a ubiquitinylated (Ub) protein of interest: a TEM (a first binding region).

FIGS. 1A, 1B, 1C, 1D, and 1E are illustrations of different embodiments of the chimeric molecules provided herein, having a first binding domain which binds to ubiquitinylated protein of interest, a linker, and a second binding domain which binds to a deubiquitinating enzyme (DUB). FIG. 1A illustrates an embodiment of a chimeric molecule provided herein, directly bound to both a deubiquitinating enzyme and to an ubiquitinylated protein of interest. FIG. 1B illustrates an embodiment of a chimeric molecule provided herein, directly bound to a deubiquitinating enzyme and indirectly bound to an ubiquitinylated protein of interest. FIG. 1C illustrates an embodiment of a chimeric molecule provided herein, indirectly bound to a deubiquitinating enzyme and directly bound to an ubiquitinylated protein of interest. FIG. 1D illustrates an embodiment of a chimeric molecule provided herein, wherein the chimeric molecule has a rigid linker and directly binds to both a deubiquitinating enzyme and an ubiquitinylated protein of interest. FIG. 1E illustrates an embodiment of a chimeric molecule provided herein, wherein the chimeric molecule has a flexible linker and directly binds to both a deubiquitinating enzyme and an ubiquitinylated protein of interest.

FIG. 2 is an illustration of an embodiment of the chimeric molecules provided herein, having a DUB engagement motif (DEM) which bind to a DUB enzyme, a flexible linker (LINK), and a TAR engagement motif (TEM), which binds to ubiquitinylated (Ub) protein of interest.

FIG. 3 is an illustration of an embodiment of the chimeric molecules provided herein, outside and inside a cell, and in different stages of activity within the cell. The chimeric molecule in Phase #1 is unbound and outside a cell. The chimeric molecule in Phase #2 has entered the cell and has bound to a cellular DUB enzyme. The chimeric molecule in Phase 3 is inside the cell and has bound to both a cellular DUB enzyme and to an ubiquitinylated protein of interest. The chimeric molecule in Phase #4 is inside the cell, has bound to both a cellular DUB enzyme and to ubiquitinylated protein of interest, and the cellular DUB enzyme has removed one or more ubiquitin molecules (Ub) from the ubiquitinylated protein. The chimeric molecule is then recycled to bind other cellular DUB enzymes and ubiquitinylated proteins of interest.

FIG. 4 is an illustration of an embodiment of the chimeric molecules provided herein, engaging a DUB enzyme via a small-molecule and engaging a target protein via a small-molecule. Upon forming a complex, the DUB enzyme snips off part of the Ub chain carried by the target protein, thus increasing the lifespan of the target protein.

FIG. 5 is an illustration of an embodiment of the chimeric molecules provided herein, engaging a DUB enzyme via a DUB engagement motif and engaging a target ubiquitinylated (Ub) p53 protein via a RITA TAR engagement motif. Upon forming a complex, the DUB enzyme snips off the Ub chain carried by the p53 target protein, thus increasing the lifespan of the p53 target protein in a cancer cell and promoting cancer cell apoptosis.

FIG. 6 is an illustration of an embodiment in which the chimeric molecules provided herein are administered to cancer patients in combination with other molecules. A RITA molecule, which can serve as a TAR engagement motif in the chimeric molecules provided herein (FIG. 5), can be administered to block ubiquitination of p53 from one of its E3 ligases, namely MDM2, in a cancer cell, and promote cancer cell apoptosis.

It will be appreciated that for simplicity and clarity of illustration, elements shown in the figures have not necessarily been drawn to scale. For example, the dimensions of some of the elements may be exaggerated relative to other elements for clarity.

### DETAILED DESCRIPTION OF THE INVENTION

In the following detailed description, numerous specific details are set forth in order to provide a thorough understanding of the chimeric molecules. However, it will be understood by those skilled in the art that the chimeric molecules described herein may be practiced without these specific details. In other instances, well-known methods, procedures, and components have not been described in detail so as not to obscure the chimeric molecules described herein.

Ubiquitin (Ub) is a highly conserved globular 76-residue eukaryotic protein found in the cytoplasm and nucleus of cells. Ubiquitin exists both as a monomer and as isopeptide-linked polymers known as poly-ubiquitin chains.

Ubiquitin can be covalently attached to lysine residues on polypeptide substrates through the sequential action of three enzymes: an ubiquitin activation enzyme (E1); an ubiquitin-conjugating enzyme (E2); and an ubiquitin ligase (E3), that catalyzes transfer of ubiquitin to substrates. The human genome encodes 2 E1s, 37 E2s, and >600 E3 ubiquitin ligases. Ubiquitin contains seven lysine residues (K6, K11, K27, K29, K33, K48, K63) that, together with its N-terminus methionine (Met1), can serve as secondary attachment points to make diverse polyubiquitin chains with different structures and functions. Ubiquitination has classically been ascribed to targeting cytosolic proteins for degradation by the proteasome. In contrast, ubiquitination of membrane proteins can lead to more nuanced outcomes including regulating protein trafficking/sorting, stability, and/or function.

The type and number of poly-ubiquitin chains that are conjugated to a target is highly regulated to generate distinct signals that affect different physiological processes. This versatility arises from the fact that not only can targets be mono-ubiquitinylated or poly-ubiquitinylated, but also that different types of poly-ubiquitin chains are formed.

The solved structures of all known Ub chains are unique, strongly suggesting that the formation and hydrolysis of each linkage is catalyzed by a specific set of conjugation enzymes and DUBs. More recently, novel Ub chains have been identified and these include non-degradable “forked” chains with heterogeneous linkages. Ubiquitination has been associated with inherited disorders such as cystic fibrosis, cardiac arrhythmias, epilepsy, and neuropathic pain, as well as infectious disease, contributing to the pathogenic lifecycle of diverse viral and bacterial pathogens.

### Survival-Targeting Chimeric (SURTAC) Molecules

Herein provided are synthetic, multi-domain, multi-functional, chimeric molecules, carefully designed to allow external manipulation of cellular protein levels. In one embodiment, the chimeric molecules provided herein are designed and targeted to remove one or more ubiquitin molecules from specific predetermined populations of proteins within cells.

In one embodiment, the chimeric molecules provided herein are able to recruit cellular deubiquitinating enzymes (DUBs) and specifically bind to target proteins that are labeled or tagged by one or more ubiquitin (Ub) molecules. The binding between the chimeric molecules provided herein and the DUBs or the ubiquitinylated proteins may be direct, or indirect. Indirect binding may be through one intermediate molecule, or by a series or chain of intermediate molecules. In one embodiment, the dual binding of both effector deubiquitinating enzymes and ubiquitinylated protein substrates results in the cleaving of one or more ubiquitin molecules from the protein substrates.

In some embodiments, cleavage comprises cleavage of a Ub-Ub bond. In some embodiments, cleavage comprises cleavage of a Ub-protein bond. In some embodiments, cleavage comprises enhanced cleavage of a Ub-Ub bond compared with cleavage of a Ub-protein bond.

In one embodiment, the removal of ubiquitin(s) from the protein substrates may be partial, i.e. the proteins disengage from the chimeric molecules provided herein with a shorter Ub chain than the Ub chain with which they were bound. In another embodiment, the removal of ubiquitin(s) may be complete, i.e. the proteins disengage from the chimeric molecules provided herein are free of any Ub molecule. In either case, the propensity of the resulting partly or completely deubiquitinated proteins to undergo UPS-related protein degradation is considerably decreased, if not nullified.

Abbreviations used throughout the Figures and the Description herein include the following: ubiquitin: Ub; a deubiquitinating enzyme: a DUB; a DUB engagement motif, which may bind to a deubiquitinating enzyme (DUB): a DEM; a linker: LINK; a target polypeptide, which may be a ubiquitinylated (Ub) protein of interest: a TAR; and a TAR engagement motif, which may bind to a ubiquitinylated (Ub) protein of interest: a TEM.

A skilled artisan would appreciate that a first binding domain described herein comprises a TAR engagement motif (TEM), wherein in certain embodiments the terms “first binding domain” and “TEM” may be used interchangeably, have the same meanings and qualities. Further, the skilled artisan would appreciate that a second binding domain described herein comprises a DUB engagement motif (DEM), wherein in certain embodiments the terms “second binding domain” and “DEM” may be used interchangeably, have the same meanings and qualities.

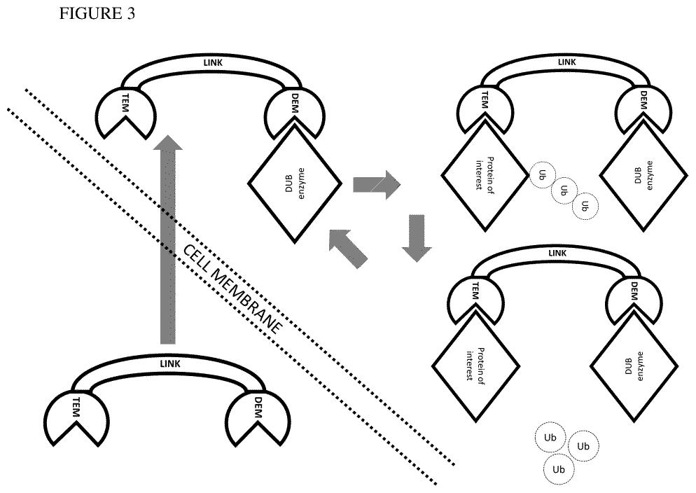

FIG. 4 illustrates an embodiment of the use of the SURTAC molecules provided herein, in which the chimeric molecule comprises two non-inhibitory small molecules that bind to their respective targets, a DUB enzyme and an ubiquitinylated protein, thereby allowing the DUB enzyme to partially deubiquitinate the ubiquitinylated protein. In some embodiments, a chimeric molecule comprising a first binding domain (a TAR engagement motif (TEM)), which in some embodiments may be a non-inhibitory small molecule, and a second binding domain (a DUB engagement motif (DEM)), which may in certain embodiments be a non-inhibitory small molecule, binds its respective targets, a DUB enzyme and a ubiquitinylated protein, allowing the DUB enzyme to partially deubiquitinate the ubiquitinylated protein.

To achieve their designated activity, the chimeric molecules provided herein are carefully designed. First, their size is kept to a minimum to allow ease of manufacture and superior cell membrane permeability. Secondly, the chimeric molecules provided herein must simultaneously target at least two naturally-occurring cellular proteins, one being an ubiquitinylated protein and the other being a DUB enzyme which is capable of partly or completely deubiquitinates the ubiquitinylated protein. To allow simultaneous binding of two different target proteins, the chimeric molecules provided herein have two different binding domains, each targeting a different target protein. Thirdly, to allow the DUB enzyme to perform its action on the ubiquitinylated protein, the two binding domains are spatially arranged to bring the enzyme and protein to sufficient proximity.

In some embodiments, an ubiquitinylated target polypeptide is cytosolic. In some embodiments, an ubiquitinylated target polypeptide is a membrane bound polypeptide. In some embodiments, an ubiquitinylated target polypeptide is a cell surface polypeptide. In some embodiments, an ubiquitinylated target polypeptide is associated with a cell surface polypeptide.

In one embodiment, the chimeric molecules provided herein may be used in therapy and research, both in-vivo and ex-vivo. A non-limiting example of an application of the chimeric molecules provided herein is decreasing, if not cancelling, unwanted degradation of functional or partly-functional proteins in diseased cells, where such a degradation aggravates the condition of the cells or of a patient carrying these cells.

Cancer cells have developed diverse mechanisms to neutralize functional tumor suppressor proteins. One of the simplest and most effective strategies is to destroy functional tumor suppressor proteins by tagging the proteins with Ub molecules. Thus, the etiology of many cancers involves unwanted degradation of functional tumor suppressor proteins, such as p53. Viruses such as human papillomaviruses 16 and 18 also use the same mechanism to prevent the infected cell from becoming apoptotic. Thus, in one embodiment, the chimeric molecules provided herein may be used to decrease UPS-related degradation of functional proteins, e.g. tumor suppressor proteins.

While in the case of tumor suppressor protein, the functional protein is tagged for degradation by disease-promoting agents, there are cases where it is beneficial to prevent the degradation of partly-functional protein, e.g. where a fully functional protein is not available to the cell. In some embodiments, preventing the degradation of a partially functioning protein may be beneficial in a subject suffering from a disease or condition, wherein prevention of degradation slows or halts the progress of the disease or condition. In some embodiments, reducing the degradation of a partially functioning protein may be beneficial in a subject suffering from a disease or condition, wherein prevention of degradation slows or halts the progress of the disease or condition. A non-limiting example of a partially functioning protein comprises a cystic fibrosis receptor channel (CFTR channel) polypeptide), wherein a wild-type CFTR polypeptide is not available.

Proteins may misfold but do not necessarily lose all their activity. Nevertheless, such proteins are rapidly destroyed by the cell. Diseases related to protein misfolding are caused by the unwanted, but natural, degradation of partially functional protein. The most common mutation in cystic fibrosis is a deletion of a single residue, phenylalanine, at position 508. The DF508 mutant (termed ΔF508) is recognized in the ER as misfolded and is degraded by the proteasome, despite retaining significant function compared to the wild-type, fully functional protein. Thus, in one embodiment, the chimeric molecules provided herein may be used to decrease UPS-related degradation of partly-functional proteins, e.g. misfolded proteins.

An additional non-limiting example of an application of the chimeric molecules provided herein is in basic cell research. By introducing the chimeric molecules provided herein to cells in culture, one can artificially increase the cellular level of a protein of interest. Such manipulation may be useful in e.g. elucidating the cells' response to elevated levels of the protein, or in elucidating signal-transduction pathways in which the protein is involved. Thus, in one embodiment, the chimeric molecules provided herein may be used to increase the cellular level of natural cellular proteins.

The ubiquitination of proteins affects proteins in at least four key aspects. First, ubiquitination can mark proteins for degradation via the proteasome. Second, ubiquitination can alter the cellular location of proteins. Third, ubiquitination can affect the activity of proteins. Fourth, ubiquitination can modulate, e.g. promotes or inhibits, protein interactions. Thus, the chimeric molecules provided herein, by performing a manipulation (e.g. decreasing) on the number of ubiquitin molecules attached to proteins of interest, can be used to interfere, modulate or control these key aspects of cellular proteins.

### Structures of Survival-Targeting Chimeric (SURTAC) Molecules

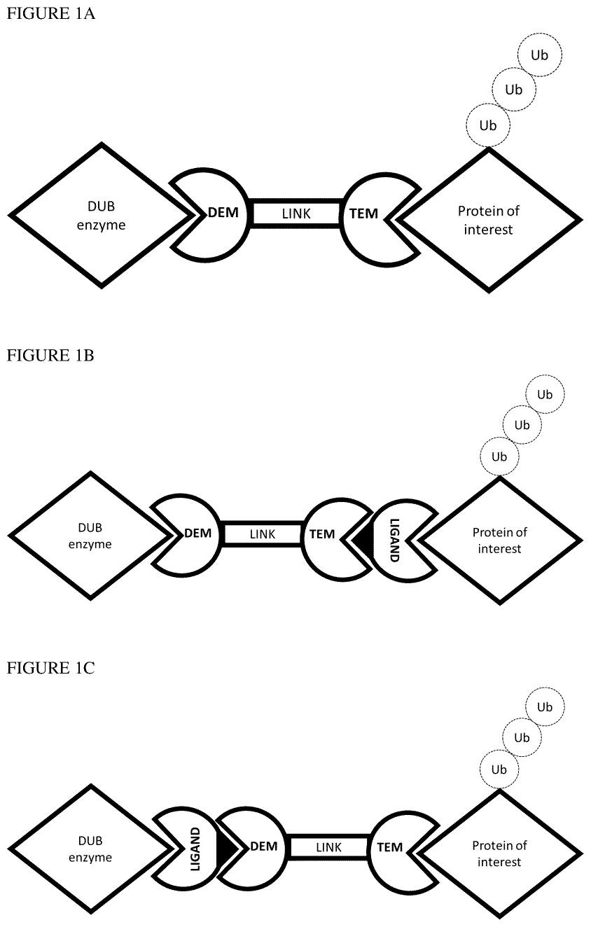

The general structure of the chimeric molecules provided herein comprises at least a first binding domain, a second binding domain, and a linker domain, wherein each of these domains is a physical region of the chimeric molecules described in detail herein, each having a unique structure and/or function. Within the Figures (for example see, FIGS. 1A-E, 2, 3, and 5) for ease of identification, the general structure of the chimeric molecules provided is shown comprising at least the formula DEM-LINK-TEM, wherein each one of DEM, LINK, and TEM is a physical region of the chimeric molecules described in detail herein, each having a unique structure and/or function.

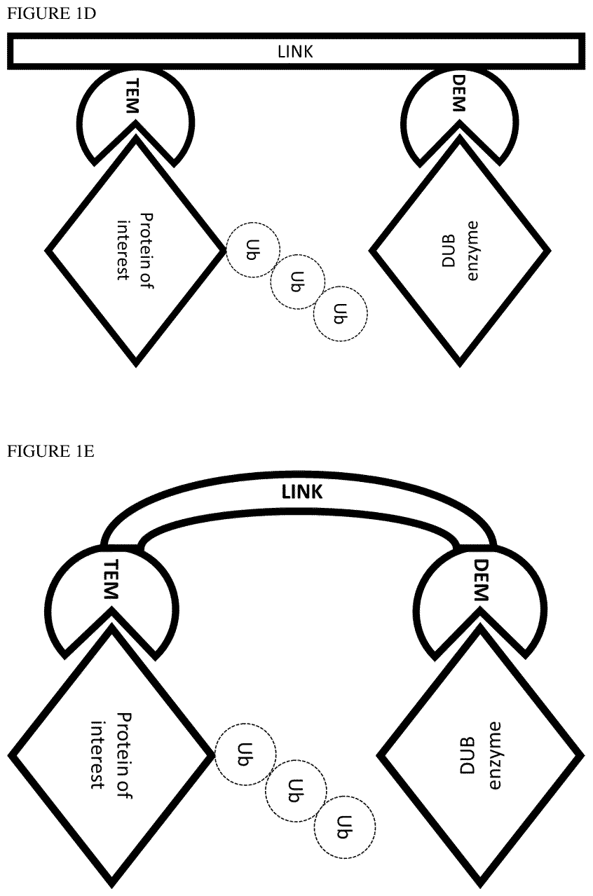

In one embodiment, the function of the first binding domain is to bind, e.g. when inside a cell, an ubiquitinylated protein. In one embodiment, the function of the second binding domain is to bind, e.g. when inside a cell, a deubiquitinating enzyme (DUB) or any other protein or enzyme capable of cleaving (a) ubiquitin, (b) bonds between ubiquitin and its substrate protein, and/or bonds between ubiquitin molecules in the same ubiquitin chain. In one embodiment, the function of the linker domain is connecting, either covalently or otherwise, the first binding domain to the second binding domain. It will be understood by those skilled in the art that the environment constituted “inside a cell” can be partly or fully reconstituted ex-vivo in any vessel, e.g. a sterile disposable laboratory tube. Non-limiting examples of certain embodiments of the chimeric molecules provided herein are presented in FIGS. 1A, 1B, 1C, 1D, 1E, and 2.

It will be understood by those skilled in the art that the chimeric molecules provided herein may comprise multiple first binding domains, multiple second binding domains, multiple linker domains, or any combinations thereof.

A skilled artisan would appreciate that the term “motif” used throughout, may in some embodiments be used interchangeable with the term “domain”, having all the same qualities and measures. The use of the term “domain” is not in any way meant to infer or limit that a specific region of the chimeric molecule is a peptide or polypeptide.

In some embodiments, a “domain” comprises a small molecule or an active portion thereof. In some embodiments, a “domain” comprises a peptide. In some embodiments, a “domain” comprises a polypeptide or a portion thereof. In some embodiments, a “domain” comprises a protein or an active portion thereof.

A skilled artisan would recognize that the first binding domain, the second binding domain, and the linker domain encompass discrete regions of the chimeric molecule described herein, and can be distinctively identified by their physical and functional properties as disclosed herein.

In some embodiments, the DUB engagement motif is in charge of recruiting (e.g. identifying and binding in a specific manner) a deubiquitinating enzyme. For establishing a connection between the chimeric molecules provided herein and deubiquitinating enzymes, the DUB engagement motif comprises a binding site which specifically binds to deubiquitinating enzymes. The deubiquitinating enzymes targeted by the DEM binding site may be any deubiquitinating enzymes, or any defined sub-category of deubiquitinating enzymes. The binding site may comprise a molecule which specifically recognizes the deubiquitinating enzyme(s), such as an antibody or a fragment thereof.

As described herein, the first binding domain and the second binding domain are spatially arranged to bring the DUBs and ubiquitinylated proteins to sufficient proximity to allow the DUBs to perform their action on the bound ubiquitinylated proteins. As the size and volume of DUBs and ubiquitinylated proteins are varied, the relative orientation of the first binding domain, the second binding domain, and the linker domain to each other is addressed when designing a particular embodiment of the chimeric molecules provided herein. In general, the chimeric molecules provided herein, and specifically the relative orientation of these three domains is configured to allow the DUBs bound by the second binding domain to deubiquitinate the ubiquitinylated protein bound by the first binding domain. It will be understood by those skilled in the art that functional molecules provided herein are those which allow the DUBs bound by the second binding domain to deubiquitinate the ubiquitinylated protein bound by the first binding domain.

In one embodiment, the chimeric molecules provided herein are designed to specifically bind various cellular proteins, e.g. ubiquitin proteases and ubiquitinylated proteins. In some embodiment, a chimeric molecule provided herein binds to an intracellular protein. In some embodiment, a chimeric molecule provided herein binds to an extracellular protein.

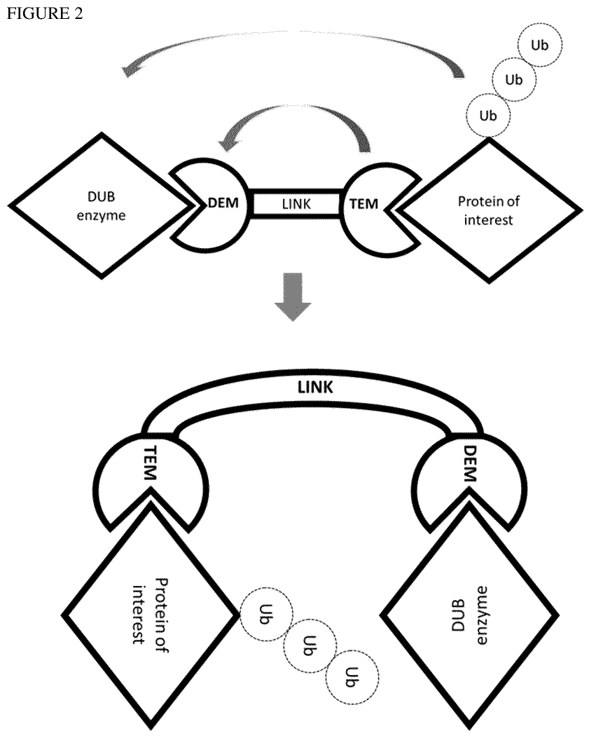

In certain embodiments, the chimeric molecule provided herein is bound to the ubiquitinylated protein. In certain embodiments, the chimeric molecule provided herein is bound to the ubiquitin protease. In certain embodiments, the chimeric molecule provided herein is bound to both the ubiquitinylated protein and the ubiquitin protease. In certain embodiments, the chimeric molecule provided herein is bound to the ubiquitinylated protein, to the ubiquitin protease, or to both the ubiquitinylated protein and the ubiquitin protease. A non-limiting example of one embodiment of the chimeric molecules provided herein, in which the chimeric molecule enters a cell, binds an ubiquitinylated protein and an ubiquitin protease, and releases the deubiquitinated protein, is presented in FIG. 3.

In certain embodiments, the chimeric molecules provided herein do not inhibit the activity of the ubiquitinylated protein and/or the activity of the ubiquitin protease during and/or after de-ubiquitination. In certain embodiments, the chimeric molecules provided herein do not inhibit the activity of the ubiquitinylated protein during and after de-ubiquitination. In certain embodiments, the chimeric molecules provided herein do not inhibit the activity of the ubiquitin protease during and after de-ubiquitination. In certain embodiments, the chimeric molecules provided herein do not inhibit the activity of the ubiquitinylated protein and the activity of the ubiquitin protease during and after de-ubiquitination.

In other embodiments, the chimeric molecules provided herein inhibit the activity of the ubiquitinylated protein and/or the activity of the ubiquitin protease. In certain embodiments, the chimeric molecules provided herein inhibit the activity of the ubiquitinylated protein during and/or after de-ubiquitination. In certain embodiments, the chimeric molecules provided herein inhibit the activity of the ubiquitin protease. In certain embodiments, the chimeric molecules provided herein partially inhibit the activity of the ubiquitinylated protein before and/or after deubiquitylation. In certain embodiments, the chimeric molecules provided herein partially inhibit the activity of the ubiquitin protease.

In certain instances, wherein a chimeric molecule partially or fully inhibits the ubiquitin protease activity and/or partially or fully inhibits the activity of the ubiquitinylated protein there may still be benefit in use of the chimeric molecule. For example, in certain embodiments a chimeric molecule may bring a DUB and an Ub-protein within functional range, independent of its effect on the DUB activity or Ub-protein activity. The chimeric molecule in certain embodiments, would be displaced, and the DUB would be in position to cleave Ub molecules from the Ub-protein. Therefore, use of the chimeric molecule would effectively maintain or increase the expected half-life of the Ub-protein.

In one embodiment, the chimeric molecules provided herein have an intrinsic capability of penetrating membranes, and in particular cell membranes. In one embodiment, the chimeric molecules provided herein do not target any particular cell population. However, promiscuous cell entry may be problematic in-vivo, especially during systemic administration. Thus, in another embodiment, the chimeric molecules provided herein may further comprise a third binding domain that specifically targets antigen(s) presented by a defined cell population. The third binding domain may comprise a molecule which specifically recognizes the cell-presented antigen, such as an antibody or a fragment thereof. In the alternative, the third binding domain may comprise a molecule which is specifically recognized by the cell-presented antigen, such as a ligand of the cell-presented antigen. In the alternative, the third binding domain may comprise a molecule which is specifically recognized by the cell-presented antigen, such as an aptamer. It will be understood by those skilled in the art that since the third binding domain binds the cell-presented antigen outside a cell, it does not covalently-link to the cell-presented antigen after binding, without additional steps. Without being bound to any theory or mechanism, it is hypothesized that the third binding domain transiently binds to the cell-presented antigen, at least for a minimal time to allow the chimeric molecules provided herein bound to the cell-presented antigen to enter the cell. It will be understood by those skilled in the art that numerous cell-type-specific antigens are already known, such as tumor-associated-antigens and tumor-specific-antigens, with more identified each year.

In another embodiment, the intrinsic capability of the chimeric molecules provided herein to penetrate membranes, e.g. cell membranes, may be fortified by further comprising a cell-penetrating tag. While cell penetration may not be a problem when employing the chimeric molecules provided herein in-vitro, when usually detached cells or several layers of cells are researched, cell entry may be problematic in-vivo, especially when targeting multi-layer tissues or organs, such as the liver or pancreas, or as in the case of solid tumors. Thus, the chimeric molecules provided herein may further comprise a cell-penetrating tag, which increases the cell or membrane-penetrating propensity of the chimeric molecules provided herein. Without being bound to any theory or mechanism, it is hypothesized that the cell-penetrating tag transiently interacts with the membrane of cells, at least for a minimal time to allow the chimeric molecules provided herein to enter the cell. It will be understood by those skilled in the art that numerous cell-penetrating tag are already known, such as cell-penetrating peptides (CPPs), with more identified each year. Cell-penetrating tags, such as cell-penetrating peptides (CPPs), may be short peptides that facilitate cellular intake/uptake of various molecules. The chimeric molecules provided herein may be associated with the CPPs either through chemical linkage via covalent bonds or through non-covalent interactions.

A skilled artisan would appreciate that a DUB that targets a protein on the cell surface reduces or eliminates the need of a chimeric molecule described herein, to enter the cell. In some embodiments, an ubiquitinylated target polypeptide is cytosolic. In some embodiments, an ubiquitinylated target polypeptide is a membrane bound polypeptide. In some embodiments, an ubiquitinylated target polypeptide is a cell surface polypeptide. In some embodiments, an ubiquitinylated target polypeptide is associated with a cell surface polypeptide.

As the chimeric molecules provided herein are synthetic, i.e. are not found in nature, it will be understood by those skilled in the art that the chimeric molecules provided herein may be produced by any known method, e.g. in the fields of protein synthesis and organic chemistry. As such, the chimeric molecules provided herein may be produced in-vitro, and, in the alternative, in-vivo. While the chimeric molecules provided herein may be made completely or partly by amino-acids, e.g. may be peptides or proteins, the chimeric molecules provided herein may be produced by nucleic acid sequences, such as mRNA, single-stranded DNA (ssDNA) and double-stranded DNA (dsDNA), encoding the chimeric molecules provided herein.

### Binding Domains

In one embodiment, the first binding domain is in charge of recruiting (e.g. identifying and binding in a specific manner) an ubiquitinylated protein. The ubiquitinylated proteins targeted by the first binding domain may be any ubiquitinylated proteins, or any defined sub-category of ubiquitinylated proteins. In one embodiment, the first binding domain comprises a binding site which specifically binds to ubiquitinylated proteins. For example, such binding site may comprise a molecule which specifically recognizes the ubiquitinylated protein(s), such as an antibody or a fragment thereof. In the alternative, the binding site may comprise a molecule which is specifically recognized by the ubiquitinylated protein(s), such as a ligand of the ubiquitinylated protein(s). In some embodiments, the binding site may comprise a molecule which is specifically recognized by the ubiquitinylated protein(s), such as an aptamer. It will be understood by those skilled in the art that since the first binding domain binds the ubiquitinylated protein(s) inside a cell, it does not covalently link to the ubiquitinylated protein(s) after binding, without additional steps. Without being bound to any theory or mechanism, it is hypothesized that the first binding domain transiently binds to the ubiquitinylated protein(s), at least for a minimal time to allow the deubiquitinating enzyme(s) bound by the second binding domain to perform their activity on the bound ubiquitinylated protein(s) as described herein.

In another embodiment, the first binding domain may directly and specifically bind to an intermediary molecule that directly and specifically binds to the target ubiquitinylated protein. Thus, in some embodiments, as the first binding domain specifically binds to the intermediary molecule, and the intermediary molecule specifically binds to the ubiquitinylated protein, the first binding domain indirectly but specifically binds to the ubiquitinylated protein. In the alternative, more than one intermediary molecule can be employed between the first binding domain and the ubiquitinylated protein, thus again the first binding domain indirectly but specifically binds to the ubiquitinylated protein. In certain embodiments, the intermediate molecule that binds to the ubiquitinylated protein comprises an antibody or an antigen-binding fragment thereof that binds to the ubiquitinylated protein. In certain embodiments, the intermediate molecule that binds to the ubiquitinylated protein comprises a ligand of the ubiquitinylated protein. In certain embodiments, the intermediate molecule that binds to the ubiquitinylated protein comprises an aptamer the binds to the ubiquitinylated protein.

In some embodiments, an ubiquitinylated target polypeptide is a cytosolic polypeptide. In some embodiments, an ubiquitinylated target polypeptide is a membrane bound polypeptide. In some embodiments, an ubiquitinylated target polypeptide is a cell surface polypeptide. In some embodiments, an ubiquitinylated target polypeptide is associated with a cell surface polypeptide.

A non-limiting example of one embodiment of the chimeric molecules provided herein, in which the first binding domain directly binds to an intermediary molecule and the intermediary molecule directly binds to an ubiquitinylated protein of interest is presented in FIG. 1B. As would be appreciated by those skilled in the art, when component “A” specifically binds component “B”, and when component “B” specifically binds component “C”, then component “A” is specifically bound to component “C”. An “intermediary molecule” is a molecule which is specifically bound to at least two other molecules.

As would be appreciated by those skilled in the art, molecules provided herein may bind their targets, perform an action on their targets, and then release their targets. In certain embodiments, the first binding domain transiently binds to the ubiquitinylated protein and dissociates from the protein after one or more ubiquitin molecules are removed from the ubiquitinylated protein. In certain embodiments, the first binding domain recognizes the ubiquitinylated protein only in its ubiquitinylated state and does not recognize the same protein in its de-ubiquitinylated state (e.g. when all or some of the ubiquitin molecules are removed from the ubiquitinylated protein).

In one embodiment, the second binding domain is in charge of recruiting (e.g. identifying and binding in a specific manner) a deubiquitinating enzyme (DUB). The DUB targeted by the second binding domain may be any deubiquitinating enzyme, or any defined sub-category of DUB. In one embodiment, the second binding domain comprises a binding site which specifically binds to DUB. For example, such binding site may comprise a molecule which specifically recognizes the DUB, such as an antibody or a fragment thereof. In the alternative, the binding site may comprise a molecule which is specifically recognized by the DUB, such as a ligand of the DUB. In some embodiments, the binding site may comprise a molecule which is specifically recognized by the DUB, such as an aptamer. It will be understood by those skilled in the art that since the second binding domain binds the DUB inside a cell, it does not covalently link to the DUB after binding, without additional steps. Without being bound to any theory or mechanism, it is hypothesized that the second binding domain transiently binds to the DUB, at least for a minimal time to allow the deubiquitinating enzyme(s) bound by the second binding domain to perform their activity on the bound ubiquitinylated protein(s) as described herein.

In another embodiment, the second binding domain may directly and specifically bind to an intermediary molecule that directly and specifically binds to the DUB. Thus, in some embodiments, as the second binding domain specifically binds to the intermediary molecule, and the intermediary molecule specifically binds to the DUB, the second binding domain indirectly but specifically binds to the DUB. In the alternative, more than one intermediary molecule can be employed between the second binding domain and the DUB, thus again the second binding domain indirectly but specifically binds to the DUB. In certain embodiments, the intermediate molecule that binds to the ubiquitin protease comprises an antibody or an antigen-binding fragment thereof that binds to the ubiquitin protease. In certain embodiments, the intermediate molecule that binds to the ubiquitin protease comprises a ligand of the ubiquitin protease. In certain embodiments, the intermediate molecule that binds to the ubiquitin protease comprises an aptamer. A non-limiting example of one embodiment of the chimeric molecules provided herein, in which the second binding domain directly binds to an intermediary molecule and the intermediary molecule directly binds to an ubiquitin protease is presented in FIG. 1C.

In certain embodiments, the second binding domain transiently binds to the ubiquitin protease and dissociates from the ubiquitin protease when the ubiquitinylated protein is de-ubiquitinylated, e.g. when one or more ubiquitin molecules are removed from the ubiquitinylated protein. In certain embodiments, the second binding domain irreversibly binds to the ubiquitin protease and does not dissociate from the ubiquitin protease when the ubiquitinylated protein is de-ubiquitinylated. In certain embodiments, the second binding domain binds to any ubiquitin protease that cleaves ubiquitin from an ubiquitinylated protein. In certain embodiments, the second binding domain binds to an ubiquitin protease that cleaves ubiquitin from the ubiquitinylated protein bound by the first binding domain. In certain embodiments, the second binding domain binds to an ubiquitin protease that cleaves ubiquitin from the ubiquitinylated protein bound by the first binding domain when both the ubiquitin protease and the ubiquitinylated protein are not bound by the chimeric molecules provided herein. In certain embodiments, the second binding domain binds to an ubiquitin protease that cleaves ubiquitin from the ubiquitinylated protein bound by first binding domain only when both the ubiquitin protease and the ubiquitinylated protein are bound by the chimeric molecules described herein.

In certain embodiments, the first or the second binding domain can be a small molecule. In one embodiment, the small molecule is an organic compound. In one embodiment, the small molecule has a size between 0.1 nm to 10 nm across its longest axis. In another embodiment, the small molecule has a size between 0.5 nm to 5 nm across its longest axis. In one embodiment, the small molecule has a weight of 1 Dalton up to 1000 Daltons. In another embodiment, the small molecule has a weight of 1 Dalton up to 500 Daltons. In another embodiment, the small molecule has a weight of 1 Dalton up to 100 Daltons.

### Deubiquitinating Enzymes

Deubiquitinases (DUBs) are specialized isopeptidases that provide salience to ubiquitin signaling through the revision and removal of ubiquitin chains. There are over 100 human DUBs, comprising 6 distinct families: 1) the ubiquitin specific proteases (USP) family, 2) the ovarian tumor proteases (OUT) family, 3) the ubiquitin C-terminal hydrolases (UCH) family, 4) the Josephin domain family (Josephin), 5) the motif interacting with ubiquitin-containing novel DUB family (MINDY), and 6) the JAB1/MPN/Mov34 metalloenzyme domain family (JAMM). Of note, the USP family is relatively promiscuous, hydrolyzing all ubiquitin linkages, in stark contrast to the OTU family, which contains a diverse set of enzymes with distinct linkage preferences. Linkage-specific DUBs have been purified and used in cell-free in vitro assays.

For the deubiquitinating enzymes (DUBs) to perform their activity on ubiquitinylated protein(s), they comprise at least one catalytic domain. The catalytic domain is the domain that comes in contact with the ubiquitin attached to the target protein and removes it from the target protein. In one embodiment, the catalytic unit of the DUB is selective for all ubiquitin linkage types. In another embodiment, the catalytic unit is selective for particular ubiquitin linkage type.

In one embodiment, the DUB comprises a catalytic domain or other domain such as an ubiquitin-specific proteases (DUSP) domain; an ubiquitin-like (UBL) domain; a meprin and TRAF homology (MATH) domain; a zinc-finger ubiquitin-specific protease (ZnF-UBP) domain; a zinc-finger myeloid, nervy and DEAF1 (ZnF-MYND) domain; an ubiquitin-associated (UBA) domain; a CHORD-SGT1 (CS) domain; a microtubule-interacting and trafficking (MIT) domain; a rhodenase-like domain; a TBC/RABGAP domain; or a B-box domain.

In one embodiment, the DUB bound by the second domain of the chimeric molecules provided herein can be a DUB from the ubiquitin specific proteases (USP) family, the ovarian tumor proteases (OUT) family, the ubiquitin C-terminal hydrolases (UCH) family, the Josephin domain family (Josephin), the motif interacting with ubiquitin-containing novel DUB family (MINDY), or the JAB1/MPN/Mov34 metalloenzyme domain family (JAMM).

In some embodiments, a DUB comprises a deubiquitinating enzyme that is part of a larger complex. In some embodiments, a DUB comprises the larger complex within which the deubiquitinating enzyme is present. In some embodiments, a DUB comprises some of the components of the larger complex within which the deubiquitinating enzyme is present. In some embodiments, a DUB comprises at least one of the components of the larger complex within which the deubiquitinating enzyme is present. In some embodiments, a DUB comprises the catalytic subunit of a deubiquitinating enzyme. In some embodiments, a DUB comprises a catalytically active fragment of the catalytic subunit of a deubiquitinating enzyme.

In some embodiments, a DUB comprises a deubiquitinating enzyme providing a promiscuous protease activity. In some embodiments, a DUB comprises a deubiquitinating enzyme providing a specific linkage cleavage.

### Ubiquitinylated Protein

In one embodiment, the chimeric molecules provided herein are designed to bring any ubiquitinylated protein in close proximity to a deubiquitinases (DUB) so that the deubiquitinases can remove one or more ubiquitin molecules from the ubiquitinylated protein. In certain embodiments, the ubiquitinylated protein carries a mono-ubiquitin molecule. In certain embodiments, the ubiquitinylated protein carries a mono-ubiquitin molecule upon binding to the chimeric molecule described above. In certain embodiments, the ubiquitinylated protein carries a poly-ubiquitin chain. In certain embodiments, the ubiquitinylated protein carries a poly-ubiquitin chain upon binding to the chimeric molecule described above. In certain embodiments, the poly-ubiquitin chain comprises at least 2 ubiquitin molecules. In certain embodiments, the poly-ubiquitin chain comprises at least 4 ubiquitin molecules. In certain embodiments, the poly-ubiquitin chain comprises at least 6 ubiquitin molecules. In certain embodiments, the poly-ubiquitin chain comprises at least 8 ubiquitin molecules. In certain embodiments, the poly-ubiquitin chain comprises at least 10 ubiquitin molecules.

In certain embodiments, the poly-ubiquitin chain comprises 2-50 ubiquitin molecules. In certain embodiments, the poly-ubiquitin chain comprises 4-45 ubiquitin molecules. In certain embodiments, the poly-ubiquitin chain comprises 6-40 ubiquitin molecules. In certain embodiments, the poly-ubiquitin chain comprises 8-35 ubiquitin molecules. In certain embodiments, the poly-ubiquitin chain comprises 10-30 ubiquitin molecules.

In one embodiment, when the catalytic unit of the DUB is selective for all ubiquitin linkage types, the ubiquitinylated protein bound by the chimeric molecules provided herein would comprise all kinds of ubiquitinylated proteins having all kinds of ubiquitin linkage types. In another embodiment, when the catalytic unit of the DUB is selective for a particular ubiquitin linkage type, the ubiquitinylated protein bound by the chimeric molecules provided herein would only comprise certain kinds of ubiquitinylated proteins having certain kinds of ubiquitin linkage types.

In another embodiment, the ubiquitinylated protein bound by the chimeric molecules provided herein can be a non-natural target of DUB, e.g. a protein that is not known to be a substrate for the DUB. Thus, the ubiquitinylated protein bound by the chimeric molecules provided herein can be a protein that is outside of the list of currently known substrates of DUB. It can be due to the fact that certain DUB, e.g. USP5, has been proposed to work more on the ubiquitin-ubiquitin linkage rather than the ubiquitin-target protein linkage so that any protein having one or more ubiquitin-ubiquitin linkage can be a target protein of the chimeric molecules provided herein.

In one embodiment, the ubiquitinylated protein bound by the chimeric molecules provided herein can interact with ubiquitin protease USP5. Examples of such ubiquitinylated proteins include, but are not limited to, CACNA1H (Voltage-dependent T-type calcium channel subunit alpha-1H), FOXM1 (Forkhead box protein M1), MAF (Transcription factor Maf), SMURF1 (E3 ubiquitin-protein ligase SMURF1), or TRIML1 (Tripartite motif family-like protein 1).

In one embodiment, the ubiquitinylated protein bound by the chimeric molecules provided herein can interact with ubiquitin protease USP7. Examples of such ubiquitinylated proteins include, but are not limited to, UVSSA (UV-stimulated scaffold protein A), XPC (Xeroderma pigmentosum group C-complementing protein), ABL1 (Abelson tyrosine-protein kinase 1), AR (Androgen receptor), ATXN1 (Ataxin-1), CHEK1 (Serine/threonine-protein kinase Chk1), CHFR (E3 ubiquitin-protein ligase CHFR), CLSPN (Claspin), CSNK2A1 (Casein kinase II subunit alpha), DAXX (Death domain-associated protein 6), DNMT1 (DNA (cytosine-5)-methyltransferase 1), FOXO1 (Forkhead box protein O1), FOXO4 (Forkhead box protein O4), GMPS (GMP synthetase), IFNAR1 (Type I interferon receptor 1), IKBKG (I-kappa-B kinase subunit gamma), KAT5 (Histone acetyltransferase KAT5), KDM1A (Lysine-specific histone demethylase 1A), MARCHF7 (Membrane Associated Ring-CH-Type Finger 7), MDM2 (E3 ubiquitin-protein ligase Mdm2), MDM4 (Mdm2-like p53-binding protein), MEX3C (RNA-binding E3 ubiquitin-protein ligase MEX3C), MYC (Myc proto-oncogene protein), MYD88 (Myeloid differentiation primary response protein MyD88), PML (Promyelocytic leukemia protein), POLH (DNA polymerase theta), PPARG (Peroxisome proliferator-activated receptor gamma), PTEN (Phosphatidylinositol 3,4,5-trisphosphate 3-phosphatase and dual-specificity protein phosphatase), RAD18 (E3 ubiquitin-protein ligase RAD18), RARA (Retinoic acid receptor alpha), RB1 (Retinoblastoma-associated protein), RELA (Transcription factor p65), RNF168 (E3 ubiquitin-protein ligase RNF168), RNF220 (E3 ubiquitin-protein ligase RNF220), SKP1 (S-phase kinase-associated protein 1), TP53 (Cellular tumor antigen p53), TRAF6 (TNF receptor-associated factor 6), TRIP12 (E3 ubiquitin-protein ligase TRIP12), or TRRAP (Transformation/transcription domain-associated protein).

In one embodiment, the ubiquitinylated protein bound by the chimeric molecules provided herein can interact with ubiquitin protease USP10. Examples of such ubiquitinylated proteins include, but are not limited to, AR (Androgen receptor), ATM (Serine-protein kinase ATM), CFTR (Cystic fibrosis transmembrane conductance regulator), EIF4G1 (Eukaryotic translation initiation factor 4 gamma 1), MSH2 (DNA mismatch repair protein Msh2), PRKAA1 (5′-AMP-activated protein kinase catalytic subunit alpha-1), PTEN (Phosphatase and tensin homolog), or TBX21 (T-box transcription factor TBX21).

In some embodiments, the ubiquitinylated protein bound to a chimeric molecule described herein is a known target of an ubiquitin protease bound to the same chimeric molecule, for example, the ubiquitinylated protein is known to be a substrate for the DUB. In some embodiments, the ubiquitinylated protein bound to a chimeric molecule described herein is a non-natural target of an ubiquitin protease bound to the same chimeric molecule, for example, but not limited to, the ubiquitinylated protein is not known to be a substrate for the DUB.

In another embodiment, the ubiquitinylated protein is a known target of an ubiquitin protease comprising USP5, or USP7, or USP10. In another embodiment, the ubiquitinylated protein is a non-natural target of the ubiquitin protease, for example, but not limited to wherein the ubiquitinylated protein is not known to be a substrate for USP5, or USP7, or USP10.

### Linker Domain

The first and/or second binding domains may be linked to the linker domain directly, indirectly, covalently, non-covalently, rigidly and/or flexibly. In some embodiments, the binding domain may be linked to the linker domain directly by a rigid covalent bond. In some embodiments, a binding domain may be linked to the linker domain directly by a covalent bond. In some embodiments, a binding domain may be linked to the linker domain directly by a flexible, covalent bond. In some embodiments, the binding domain may be linked to the linker domain directly by a rigid non-covalent bond. In some embodiments, a binding domain may be linked to the linker domain directly by a non-covalent bond. In some embodiments, a binding domain may be linked to the linker domain directly by a flexible, non-covalent bond. In some embodiments, one binding domain may be linked to the linker domain by a covalent bond and while the second binding domain may be linked by a non-covalent bond.

A skilled artisan would appreciate, that the linker domain has to be sufficiently flexible to successfully bring the DUB and the targeted ubiquitinylated protein together efficiently. In some embodiments, the linker domain comprises a linker rigid enough to prevent too much movement and entropy issues. In some embodiments, the length of the linker domain comprises a length that provides for bringing the DUB and the targeted ubiquitinylated protein together efficiently. In some embodiments, the combination of flexibility and length of the linker domain provide for bringing the DUB and the targeted ubiquitinylated protein together efficiently. The skilled artisan would appreciate that the linker domain, therefore, should be efficient in both size and flexibility.

In one embodiment, the linker domain is in charge of connecting the first binding domain to the second binding domain. It will be understood by those skilled in the art that the connection between the first binding domain and the second binding domain may be achieved in numerous manners. For example, the connection may be covalent or non-covalent. It will be understood by those skilled in the art that the linker domain may be a direct covalent bond between the first binding domain and the second binding domain. In one embodiment, covalent linkage includes simple single, double or triple covalent bonds between atoms in the first binding domain and the second binding domain, either directly, or indirectly through a series of atoms and covalent bonds. In one embodiment, non-covalent linkage includes all forms of non-covalent inter-molecule interactions, including but not limited to, electrostatic interactions, hydrogen-bond interaction, Van der Waals forces, hydrophobic interactions and hydrophilic interactions.

In certain embodiments, the linker domain is a single amino acid. In certain embodiments, the linker domain comprises a peptide. In certain embodiments, the peptide comprises 2-50 amino acids. In certain embodiments, the peptide comprises 4-10 amino acids. In some embodiments, the peptide comprises 4, 5, 6, 7, 8, 9, or 10 amino acids. In some embodiments, the peptide comprises 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids.

In certain embodiments, the linker domain comprises a small molecule. In certain embodiments, the small molecule is an organic compound. In certain embodiments, the small molecule is a synthetic non-naturally occurring compound. In one embodiment, the linker domain may be a small organic molecule of a low molecular weight of up to 1,000 Daltons, with a size of 10 nm or less. In another embodiment, the linker domain may be a short peptide, containing for example, approximately 100 or less amino acids.

In certain embodiments, the linker domain is configured to position the ubiquitin protease in proximity to the ubiquitinylated protein. As would be appreciated by those skilled in the art, the proximity or distance between the ubiquitin protease to the ubiquitinylated protein necessary for the ubiquitin protease to de-ubiquitinate the ubiquitinylated protein would vary depending on the protease/protein combinations.

In certain embodiments, the distance of the ubiquitin protease to the ubiquitinylated protein is 20 Å to 1 Å. In certain embodiments, the distance of the ubiquitin protease to the ubiquitinylated protein is 20 Å or less. In certain embodiments, the distance of the ubiquitin protease to the ubiquitinylated protein is 15 Å or less. In certain embodiments, the distance of the ubiquitin protease to the ubiquitinylated protein is 10 Å or less. In certain embodiments, the distance of the ubiquitin protease to the ubiquitinylated protein is 5 Å or less. In certain embodiments, the distance between an ubiquitin protease to an ubiquitinylated protein is such that the ubiquitin protease, despite not deubiquitinating the ubiquitinylated protein when both are not bound by the chimeric molecules provided herein, does deubiquitinate the ubiquitinylated protein when both are bound by the chimeric molecules provided herein.

In some embodiments, the linker is between 5 and 20 carbon atoms long. In some embodiments, the linker is between 2 and 18 carbon atoms long. In some embodiments, the linker is between 2 and 20 carbon atoms long. In some embodiments, the linker is between 5 and 10 atoms long. In some embodiments, the linker is between 10 and 15 atoms long. In some embodiments, the linker is between 15 and 20 atoms long. In some embodiments, the linker is between 10 and 20 atoms long. In some embodiments, the linker is 2 atoms long, 3 atoms long, 4 atoms long, 5 atoms long, 6 atoms long, 7 atoms long, 8 atoms long, 9 atoms long, 10 atoms long, 11 atoms long, 12 atoms long, 13 atoms long, 14 atoms long, 15 atoms long, 16 atoms long, 17 atoms long, 18 atoms long, 19 atoms long, or 20 atoms long.

One of ordinary skill in the art would readily recognize that various kinds of linkers generally known in the art could be incorporated into the chimeric molecules provided herein, for example, see WO 2014/108452, WO 2011/008260 etc, which are incorporated herein in their entirety. Furthermore, one of ordinary skill in the art would also recognize that the various linkers employed in the bifunctional proteolysis targeting chimeric (PROTAC) compounds could be incorporated into the chimeric molecules provided herein, for example, see WO 2016/197114, U.S. Pat. Nos. 9,632,089, 9,938,264 etc, which are incorporated herein in their entirety.

In some embodiments, the linker comprises a polyethylene glycol. In some embodiments, linker comprises an alkyl. In some embodiments, the linker comprises an alkenyl. In some embodiments, the linker comprises an alkyl phosphate. In some embodiments, the linker comprises an alkyl siloxane. In some embodiments, the linker comprises an epoxy. In some embodiments, the linker comprises an acylhalide. In some embodiments, the linker comprises a glycidyl. In some embodiments, the linker comprises a carboxylate. In some embodiments, the linker comprises an anhydride.

In some embodiments, the linker comprises a C1 to C18 alkylene substituted with at least one carboxyl moiety. In certain embodiments, the linker may be derived from a C1 to C18 alkylene substituted with at least one carboxyl moiety. In certain embodiments, the linker may be derived from an amino acid of natural or synthetic source having a chain length of between 2 and 18 carbon atoms (polypeptide), or an acyl halide of said amino acid. Non-limiting examples for such amino acids are 18-amino octadecanoic acid and 18-amino stearic acid.

In some embodiments, a linker comprises an amino acid of natural or synthetic source having a chain length of between 2 and 18 carbon atoms (polypeptide), or an acyl halide of said amino acid. In some embodiments, a linker comprises an amino acid of natural or synthetic source having a chain length of 2, 3, 4, 5, 6, 7, 8, 9, 10, 11, 12, 13, 14, 15, 16, 17, or 18 carbon atoms (polypeptide), or an acyl halide of said amino acid.

In some embodiments, the linker comprises a C1 to C18 alkylene. This linker may, in some embodiments, be derived from a di-halo alkylene. In some embodiments, a linker comprises a C1 alkylene, a C2 alkylene, a C3 alkylene, a C4 alkylene, a C5 alkylene, a C6 alkylene, a C7 alkylene, a C8 alkylene, a C9 alkylene, a C10 alkylene, a C11 alkylene, a 12 alkylene, a C13 alkylene, a C14 alkylene, a 15 alkylene, a C16 alkylene, a C17 alkylene, or a C18 alkylene.

In some embodiments, the linker is an aromatic group derived from non-limiting examples of 4,4-biphenol, dibenzoic acid, dibenzoic halides, dibenzoic sulphonates, terephthalic acid, terephthalic halides, and terephthalic sulphonates.

As described in WO 2014/108452 and incorporated herein in its entirety, in some embodiments, a linker comprises a linking group comprising a length of 6-16 atoms in shortest length having the formula —(CH₂)n-(R₁CH₂CH₂)m(OCH₂)qCONH—, n is 0-6, m is 2-10, q is 0 or 1, each R₁ is independently —O—, —NH—, —N(C1-3 alkyl)-, or a 4-6 membered heterocyclyl group containing 2 N atoms linked to the carbons in the chain via the ring N atoms (optionally substituted by oxo).

In some embodiments, the linker comprises (CH₂)₄(OCH₂CH₂)₃OCH₂ONH; (CH₂)₄(OCH₂CH₂)₂OCH₂CONH; (CH₂)₄(OCH₂CH₂)₄OCH₂CONH; (CH₂)₅N(CH₃)CH₂CH₂(OCH₂CH₂)₃CONH; (CH₂)₅N(CH₃)CH₂CH₂(OCH₂CH₂)₂CONH; (CH₂)₅

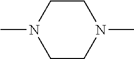

CH₂CH₂(OCH₂CH₂)₂OCHCONH; (CH₂)₆(OCH₂CH₂)₂OCH₂CONH;

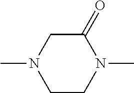

or (CH₂)₄CH₂CH₂OCH₂CH₂OCH₂CONH. WO 2014/108452 disclosure directed to these link domains is incorporated herein in its entirety.

As described in WO 2016/197114 and incorporated herein in its entirety, in some embodiments, the linker domain comprises a group comprising one or more covalently connected structural units of A (e.g. -A1 . . . Aq-), wherein q is an integer greater than or equal to 0. In some embodiments, q is an integer from 1 to 100, 1 to 90, 1 to 80, 1 to 70, 1 to 60, 1 to 50, 1 to 40, 1 to 30, 1 to 20, or 1 to 10. In certain embodiments, A1 to Aq are, each independently, a bond, CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C\hat{ }C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₃₋₁₁heterocyclyl optionally substituted with 0-6 R^{L1} and/or R^{L2} groups, aryl optionally substituted with 0-6 R^{L1} and/or R^{L2} groups, heteroaryl optionally substituted with 0-6 R^{L1} and/or R^{L2} groups, wherein R^{L1} or R^{L2}, each independently, can be linked to other A groups to form cycloalkyl and/or heterocyclyl moeity which can be further substituted with 0-4 R^{L5} groups; and wherein R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)CH(C₈alkyl), C(C₈alkyl)C(C₈alkyl), Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl)SO₂N(C₁₋₈alkyl)₂, NHSO₂NH(C₁₋₈alkyl), NHSO₂N(C₁₋₈alkyl)₂, or NHSO₂NH₂.

In another embodiment, the linker domain may comprise an optionally substituted (poly)ethylene glycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, or optionally substituted alkyl groups inter-dispersed with optionally substituted, O, N, S, P or Si atoms. In certain embodiments, the linker is substituted with an aryl, phenyl, benzyl, alkyl, alkylene, or heterocycle group.

In some embodiments, a linker domain comprises a structure such as of polyethylene glycol, an aromatic group, an alkyl, an alkenyl, an alkyl phosphate, an alkyl siloxane, an epoxy, an acyl halide, a glycidyl, a carboxylate, and an anhydride. In some embodiments, a linker domain comprises a structure comprising a polyethylene glycol. In some embodiments, a linker domain comprises a structure comprising an aromatic group. In some embodiments, a linker domain comprises a structure comprising an alkyl. In some embodiments, a linker domain comprises a structure comprising an alkenyl. In some embodiments, a linker domain comprises a structure comprising an alkyl phosphate. In some embodiments, a linker domain comprises a structure comprising an alkyl siloxane. In some embodiments, a linker domain comprises a structure comprising an epoxy. In some embodiments, a linker domain comprises a structure comprising an acyl halide. In some embodiments, a linker domain comprises a structure comprising a glycidyl. In some embodiments, a linker domain comprises a structure comprising a carboxylate. In some embodiments, a linker domain comprises a structure comprising an anhydride.

In some embodiments, the linker domain may comprise one of the following linking domains:

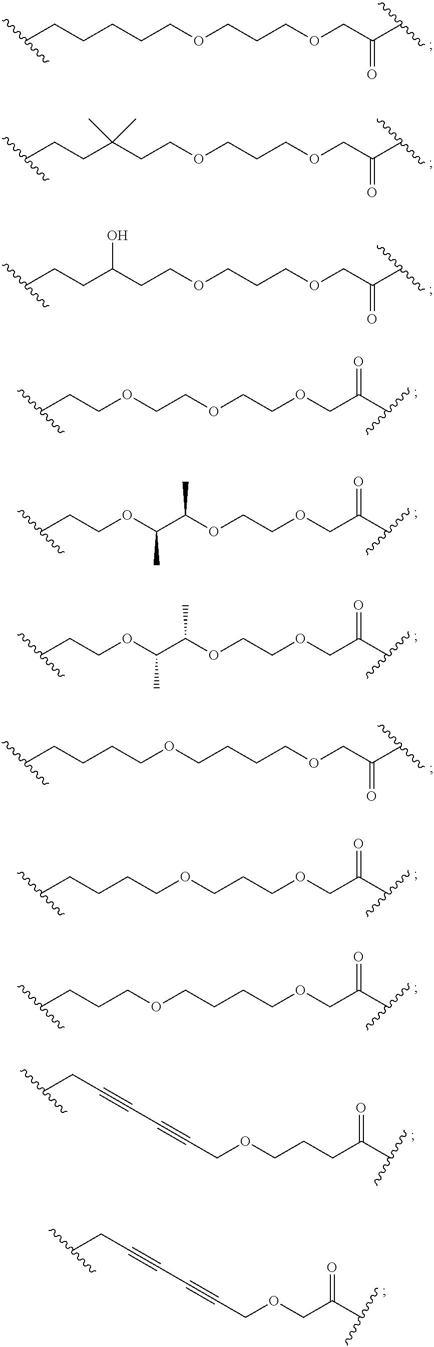

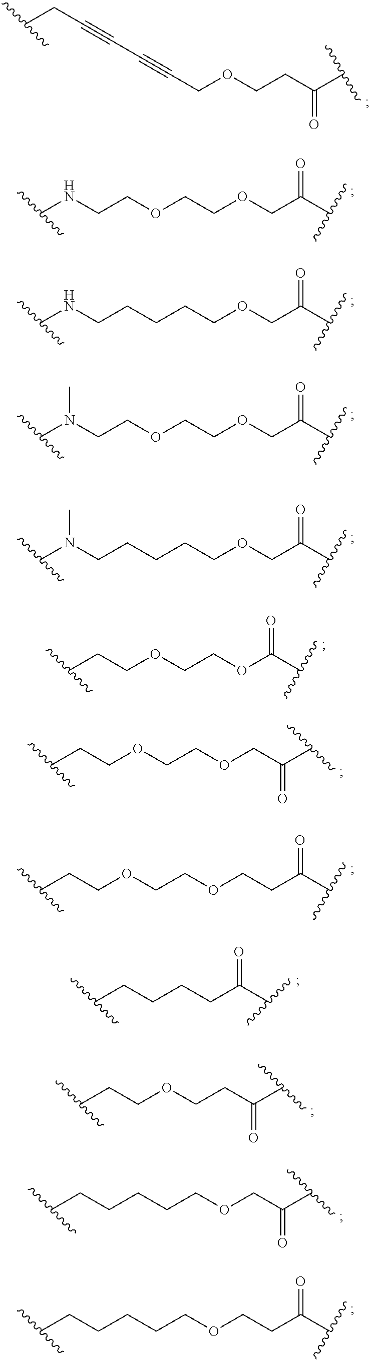

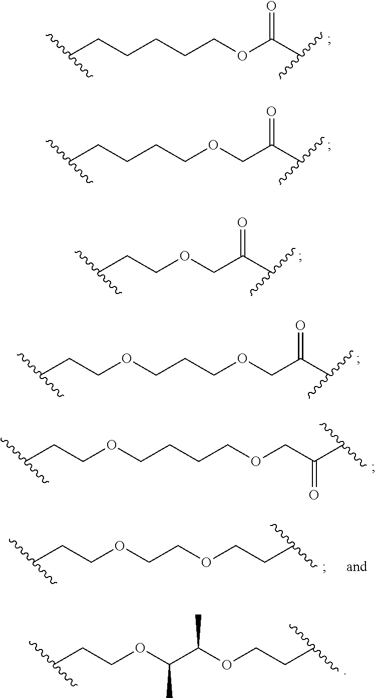

In certain embodiments, each of the linker units is independently a substituted or unsubstituted linear or branched alkyl chains of 2-50 carbon atoms, alkyl phosphate chains of 2-50 carbon atoms, alkyl ether chains of 2-50 carbon atoms (e.g. PEG, PPG of various lengths) or any combination thereof. In some embodiments, the linker comprises an optionally substituted (poly)ethylene glycol having 8 ethylene glycol units. In additional embodiments, the linker comprises an optionally substituted (poly)ethylene glycol having 11 ethylene glycol units. In additional embodiments, the linker comprises an optionally substituted (poly)ethylene glycol having 14 ethylene glycol units. In additional embodiments, the linker comprises an optionally substituted (poly)ethylene glycol having 17 ethylene glycol units.

In certain embodiments, the linker may be asymmetric. In certain embodiments, the linker may be symmetrical.

The chemistry of attachment of a linker to the binding domains include but is not limited to esters, amides, amines, hydrazide, thiols, sulfones, sulfoxides, ethers, hydroxamides, heterocycles, acetylenes, alkyls and alkenes.

### Uses of Chimeric Molecules

The design and structure of the chimeric molecules provided herein enable their use as sole active agents in all applications, either in e.g. therapy or in research. While used alone, as first line therapy, the chimeric molecules provided herein are capable of forming protein complexes between DUBs and ubiquitinylated proteins which result in a decrease of the number of ubiquitin molecules carried by the ubiquitinylated proteins. As generally disclosed herein, due to the elaborate role ubiquitination plays on cellular proteins, the chimeric molecules provided herein may be used to affect a plethora of cellular proteins and processes. It will be understood by those skilled in the art that by providing a tool to affect a key cellular regulator such as ubiquitination, many applications are envisaged based on current scientific knowledge, and more applications will become apparent as research of ubiquitination progresses.

In some embodiment, the level of ubiquitination affects the half-life of a protein of interest in a cell. In some embodiment, the level of ubiquitination affects the degradation of a protein of interest in a cell. In some embodiments, the protein of interest plays a regulatory role in a cell. In some embodiments, the protein of interest plays a regulatory role in a cellular homeostasis. In some embodiments, the protein of interest is considered a house keeping protein.

A non-limiting example of one application of the chimeric molecules provided herein is the deubiquitylation of proteins (e.g. removing all or some of ubiquitin molecules from the proteins) which are ubiquitinylated. In some embodiments, removing all or some of ubiquitin molecules maintains or increases the half-life of a protein. In some embodiments, removing all or some of ubiquitin molecules reduces the degradation of a protein. In some embodiments, maintenance or increasing the half-life of a protein, provides a benefit to a subject suffering a disease or condition. In some embodiments, decreasing degradation of a protein increases the protein's half-life. In some embodiments, preventing degradation of a protein increases the protein's half-life. In some embodiments, decreasing degradation of a protein maintains the protein's half-life. In some embodiments, preventing degradation of a protein maintains the protein's half-life. In some embodiments, decreasing the degradation of a protein, provides a benefit to a subject suffering a disease or condition. In certain embodiments, benefits may include maintenance of a regulatory function or functions performed by the protein.

In some embodiments of a method of use of the chimeric molecules provided herein for deubiquitylation of proteins (e.g. removing all or some of ubiquitin molecules from the proteins) which are ubiquitinylated, the method occurs in vitro. In some embodiments of a method of use of the chimeric molecules provided herein for deubiquitylation of proteins (e.g. removing all or some of ubiquitin molecules from the proteins) which are ubiquitinylated, the method occurs in vivo.

In some embodiments, a non-limiting example of an application of a chimeric molecule described herein comprises a method for removing at least one ubiquitin molecule from a ubiquitinylated protein, comprising contacting the ubiquitinylated protein with a survival-targeting chimeric (SURTAC) molecule comprising a first binding domain, a second binding domain, and a linker domain, wherein: the first binding domain is configured to bind to an ubiquitinylated protein; the second binding domain is configured to bind to an ubiquitin protease that cleaves ubiquitin from the ubiquitinylated protein bound to the first binding domain, and the linker domain is configured to link the first binding domain to the second binding domain; thereby removing at least one ubiquitin molecule from a ubiquitinylated protein.

In some embodiments, a non-limiting example of an application of a chimeric molecule described herein comprises a method for removing at least one ubiquitin molecule from a ubiquitinylated protein, wherein said method is in vitro. In some embodiments, a non-limiting example of an application of a chimeric molecule described herein comprises a method for removing at least one ubiquitin molecule from a ubiquitinylated protein, wherein said method is in vivo.

In some embodiments, a non-limiting example of an application of a chimeric molecule described herein comprises a method for preventing or reducing the degradation of a ubiquitinylated protein, the method comprising contacting the ubiquitinylated protein with a survival-targeting chimeric (SURTAC) molecule comprising a first binding domain, a second binding domain, and a linker domain, wherein: the first binding domain is configured to bind to an ubiquitinylated protein; the second binding domain is configured to bind to an ubiquitin protease that cleaves ubiquitin from the ubiquitinylated protein bound to the first binding domain, and the linker domain is configured to link the first binding domain to the second binding domain; thereby preventing, reducing, or ameliorating the degradation of the ubiquitinylated protein.

In some embodiments, a non-limiting example of an application of a chimeric molecule described herein comprises a method for preventing or reducing the degradation of a ubiquitinylated protein, wherein said method in in vitro. In some embodiments, a non-limiting example of an application of a chimeric molecule described herein comprises a method for preventing or reducing the degradation of a ubiquitinylated protein, wherein said method in in vivo.

Chimeric (SURTAC) molecules and components thereof have been described in detail above. In some embodiments of the methods of use of the SURTAC molecules disclosed herein, the ubiquitinylated protein comprises CACNA1H (Voltage-dependent T-type calcium channel subunit alpha-1H), FOXM1 (Forkhead box protein M1), MAF (Transcription factor Maf), SMURF1 (E3 ubiquitin-protein ligase SMURF1), or TRIML1 (Tripartite motif family-like protein 1). In some embodiments of the methods of use of the SURTAC molecules disclosed herein, the ubiquitinylated protein comprises CACNA1H (Voltage-dependent T-type calcium channel subunit alpha-1H), FOXM1 (Forkhead box protein M1), MAF (Transcription factor Maf), SMURF1 (E3 ubiquitin-protein ligase SMURF1), or TRIML1 (Tripartite motif family-like protein 1), and the ubiquitin protease comprises USP5.

In some embodiments of the methods of use of the SURTAC molecules disclosed herein, the ubiquitinylated protein comprises UVSSA (UV-stimulated scaffold protein A), XPC (Xeroderma pigmentosum group C-complementing protein), ABL1 (Abelson tyrosine-protein kinase 1), AR (Androgen receptor), ATXN1 (Ataxin-1), CHEK1 (Serine/threonine-protein kinase Chk1), CHFR (E3 ubiquitin-protein ligase CHFR), CLSPN (Claspin), CSNK2A1 (Casein kinase II subunit alpha), DAXX (Death domain-associated protein 6), DNMT1 (DNA (cytosine-5)-methyltransferase 1), FOXO1 (Forkhead box protein O1), FOXO4 (Forkhead box protein O4), GMPS (GMP synthetase), IFNAR1 (Type I interferon receptor 1), IKBKG (I-kappa-B kinase subunit gamma), KAT5 (Histone acetyltransferase KAT5), KDM1A (Lysine-specific histone demethylase 1A), MARCHF7 (Membrane Associated Ring-CH-Type Finger 7), MDM2 (E3 ubiquitin-protein ligase Mdm2), MDM4 (Mdm2-like p53-binding protein), MEX3C (RNA-binding E3 ubiquitin-protein ligase MEX3C), MYC (Myc proto-oncogene protein), MYD88 (Myeloid differentiation primary response protein MyD88), PML (Promyelocytic leukemia protein), POLH (DNA polymerase theta), PPARG (Peroxisome proliferator-activated receptor gamma), PTEN (Phosphatidylinositol 3,4,5-trisphosphate 3-phosphatase and dual-specificity protein phosphatase), RAD18 (E3 ubiquitin-protein ligase RAD18), RARA (Retinoic acid receptor alpha), RB1 (Retinoblastoma-associated protein), RELA (Transcription factor p65), RNF168 (E3 ubiquitin-protein ligase RNF168), RNF220 (E3 ubiquitin-protein ligase RNF220), SKP1 (S-phase kinase-associated protein 1), TP53 (Cellular tumor antigen p53), TRAF6 (TNF receptor-associated factor 6), TRIP12 (E3 ubiquitin-protein ligase TRIP12), or TRRAP (Transformation/transcription domain-associated protein). In some embodiments of the methods of use of the SURTAC molecules disclosed herein, the ubiquitinylated protein comprises UVSSA (UV-stimulated scaffold protein A), XPC (Xeroderma pigmentosum group C-complementing protein), ABL1 (Abelson tyrosine-protein kinase 1), AR (Androgen receptor), ATXN1 (Ataxin-1), CHEK1 (Serine/threonine-protein kinase Chk1), CHFR (E3 ubiquitin-protein ligase CHFR), CLSPN (Claspin), CSNK2A1 (Casein kinase II subunit alpha), DAXX (Death domain-associated protein 6), DNMT1 (DNA (cytosine-5)-methyltransferase 1), FOXO1 (Forkhead box protein O1), FOXO4 (Forkhead box protein O4), GMPS (GMP synthetase), IFNAR1 (Type I interferon receptor 1), IKBKG (I-kappa-B kinase subunit gamma), KAT5 (Histone acetyltransferase KAT5), KDM1A (Lysine-specific histone demethylase 1A), MARCHF7 (Membrane Associated Ring-CH-Type Finger 7), MDM2 (E3 ubiquitin-protein ligase Mdm2), MDM4 (Mdm2-like p53-binding protein), MEX3C (RNA-binding E3 ubiquitin-protein ligase MEX3C), MYC (Myc proto-oncogene protein), MYD88 (Myeloid differentiation primary response protein MyD88), PML (Promyelocytic leukemia protein), POLH (DNA polymerase theta), PPARG (Peroxisome proliferator-activated receptor gamma), PTEN (Phosphatidylinositol 3,4,5-trisphosphate 3-phosphatase and dual-specificity protein phosphatase), RAD18 (E3 ubiquitin-protein ligase RAD18), RARA (Retinoic acid receptor alpha), RB1 (Retinoblastoma-associated protein), RELA (Transcription factor p65), RNF168 (E3 ubiquitin-protein ligase RNF168), RNF220 (E3 ubiquitin-protein ligase RNF220), SKP1 (S-phase kinase-associated protein 1), TP53 (Cellular tumor antigen p53), TRAF6 (TNF receptor-associated factor 6), TRIP12 (E3 ubiquitin-protein ligase TRIP12), or TRRAP (Transformation/transcription domain-associated protein), and the ubiquitin protease comprises USP7.

In some embodiments of the methods of use of the SURTAC molecules disclosed herein, the ubiquitinylated protein comprises AR (Androgen receptor), ATM (Serine-protein kinase ATM), CFTR (Cystic fibrosis transmembrane conductance regulator), EIF4G1 (Eukaryotic translation initiation factor 4 gamma 1), MSH2 (DNA mismatch repair protein Msh2), PRKAA1 (5′-AMP-activated protein kinase catalytic subunit alpha-1), PTEN (Phosphatase and tensin homolog), or TBX21 (T-box transcription factor TBX21). In some embodiments of the methods of use of the SURTAC molecules disclosed herein, the ubiquitinylated protein comprises AR (Androgen receptor), ATM (Serine-protein kinase ATM), CFTR (Cystic fibrosis transmembrane conductance regulator), EIF4G1 (Eukaryotic translation initiation factor 4 gamma 1), MSH2 (DNA mismatch repair protein Msh2), PRKAA1 (5′-AMP-activated protein kinase catalytic subunit alpha-1), PTEN (Phosphatase and tensin homolog), or TBX21 (T-box transcription factor TBX21), and the ubiquitin protease comprises USP10.

In some embodiments of the methods of use of the SURTAC molecules disclosed herein, the ubiquitinylated protein comprises a non-natural target of the ubiquitin protease.

In some embodiments of the methods of use of the SURTAC molecules disclosed herein, the ubiquitin protease comprises a USP5. In some embodiments of the methods of use of the SURTAC molecules disclosed herein, the ubiquitin protease comprises a USP7. In some embodiments of the methods of use of the SURTAC molecules disclosed herein, the ubiquitin protease comprises a USP10.

In some embodiments of the methods of use of the SURTAC molecules disclosed herein, the ubiquitin protease comprises a domain selected from the group consisting of ubiquitin-specific proteases (DUSP) domain, ubiquitin-like (UBL) domain, meprin and TRAF homology (MATH) domain, zinc-finger ubiquitin-specific protease (ZnF-UBP) domain, zinc-finger myeloid, nervy and DEAF1 (ZnF-MYND) domain, ubiquitin-associated (UBA) domain, CHORD-SGT1 (CS) domain, microtubule-interacting and trafficking (MIT) domain, rhodenase-like domain, TBC/RABGAP domain, and B-box domain, and any combination thereof

In some embodiments of the methods of use of the SURTAC molecules disclosed herein, the ubiquitin protease is from a family selected from the group consisting of ubiquitin specific proteases (USP) family, ovarian tumor proteases (OUT) family, ubiquitin C-terminal hydrolases (UCH) family, Josephin domain family (Josephin), motif interacting with ubiquitin-containing novel deubiquitinase family (MINDY), and JAB1/MPN/Mov34 metalloenzyme domain family (JAMM).

In certain embodiments of the methods of use of the SURTAC molecule disclosed herein, the half maximal effective concentration (EC₅₀) of the chimeric SURTAC molecule described above is ≤10 μM. In certain embodiments, the EC₅₀ of the chimeric SURTAC molecule described above is ≤1 μM. In certain embodiments, the EC₅₀ of the chimeric SURTAC molecule described above is ≤0.1 μM. In certain embodiments, the EC₅₀ of the chimeric SURTAC molecule described above is between 1 μM and 10 μM. In certain embodiments, the EC₅₀ of the chimeric SURTAC molecule described above is between 0.1 μM and 1 μM.

A non-limiting example of one application of the chimeric molecules provided herein is the deubiquitylation of proteins (e.g. removing all or some of ubiquitin molecules from the proteins) which are over-ubiquitinylated due to or during neoplastic transformation. As cells are seldom transformed while keeping a normal level of functional tumor-suppressor proteins, which would otherwise direct the cells to apoptosis, over-ubiquitinylation and therefore over-degradation of tumor-suppressor proteins is a hallmark of neoplastic transformation. Thus, the specific salvage of ubiquitinylated tumor-suppressor proteins by the chimeric molecules provided herein is beneficial in fighting neoplastic transformation, cancer, and metastases of a cancer.

In some embodiments, a non-limiting example of one application of the chimeric molecules provided herein is the deubiquitylation of proteins (e.g. removing all or some of ubiquitin molecules from the proteins) which are over-ubiquitinylated due to or during neoplastic transformation, wherein said application is in vivo.

Another non-limiting example of one application of the chimeric molecules provided herein is the deubiquitylation of proteins which are over-ubiquitinylated due to or during cellular infection. As cells detect being infected by parasites, they usually initiate apoptosis by increasing the levels of apoptotic proteins. As cells do not remain viable after inducing self-apoptosis, over-ubiquitinylation and therefore over-degradation of apoptotic proteins is a hallmark of infection by e.g. viruses and bacteria, such as human papillomaviruses 16 and 18. Thus, specific salvage of ubiquitinylated apoptotic proteins by the chimeric molecules provided herein is beneficial in fighting infection.

In some embodiments, another non-limiting example of one application of the chimeric molecules provided herein is the deubiquitylation of proteins which are over-ubiquitinylated due to or during cellular infection, wherein said application is in vivo.

Another non-limiting example of one application of the chimeric molecules provided herein is the deubiquitylation of proteins which are ubiquitinylated due to misfolding. As cells detect misfolded proteins, they usually tag these proteins for degradation. As cells do not distinguish between non-functional misfolded proteins and partly-functional misfolded proteins, ubiquitinylation and therefore degradation of partly-functional misfolded proteins is a hallmark of certain diseases, such as Cystic Fibrosis. Thus, specific salvage of misfolded but still functional proteins by the chimeric molecules provided herein is beneficial in fighting disease or condition caused by misfolded proteins.

In some embodiments, in another non-limiting example of one application of the chimeric molecules provided herein is the deubiquitylation of proteins which are ubiquitinylated due to misfolding, wherein said application is in vitro. In some embodiments, in another non-limiting example of one application of the chimeric molecules provided herein is the deubiquitylation of proteins which are ubiquitinylated due to misfolding, wherein said application is in vivo.

Further provided herein are methods employing the chimeric molecules provided herein in different utilities. In one embodiment, there is provided a method for removing at least one ubiquitin molecule from an ubiquitinylated protein, the method comprising contacting the ubiquitinylated protein with the chimeric molecules described herein, thereby bringing an ubiquitin protease in close proximity to the ubiquitinylated protein to remove at least one ubiquitin molecule from the ubiquitinylated protein. In some embodiments, methods employing the chimeric molecules provided herein in different utilities are in vitro methods. In some embodiments, methods employing the chimeric molecules provided herein in different utilities are in vivo methods.

Further provided herein is a method for modulating the activity of an ubiquitinylated protein, the method comprising contacting the ubiquitinylated protein with the chimeric molecules described herein. As a result, an ubiquitin protease is placed in close proximity to the ubiquitinylated protein to remove at least one ubiquitin molecule from the ubiquitinylated protein, thereby modulating the activity of the ubiquitinylated protein. In some embodiments, a method for modulating the activity of an ubiquitinylated protein comprises an in vitro method. In some embodiments, a method for modulating the activity of an ubiquitinylated protein comprises an in vivo method.

Further provided herein is a method for modulating the cellular location of an ubiquitinylated protein, the method comprising contacting the ubiquitinylated protein with the chimeric molecules described herein. As a result, an ubiquitin protease is placed in close proximity to the ubiquitinylated protein to remove at least one ubiquitin molecule from the ubiquitinylated protein, thereby modulating the cellular location of the ubiquitinylated protein.

Further provided herein is a method for modulating the interaction of an ubiquitinylated protein with another protein, the method comprising contacting the ubiquitinylated protein with the chimeric molecules described herein. As a result, an ubiquitin protease is placed in close proximity to the ubiquitinylated protein to remove at least one ubiquitin molecule from the ubiquitinylated protein, thereby modulating the interaction of the ubiquitinylated protein with another protein.

Further provided herein is a method for treating, reducing, or ameliorating cancer in a subject, the method comprising administering to the subject the chimeric molecules described herein. As a result, an ubiquitin protease is placed in close proximity to an ubiquitinylated protein to remove at least one ubiquitin molecule from the ubiquitinylated protein, thereby treating, reducing, or ameliorating cancer in the subject.

In some embodiments of a method described herein, the chimeric SURTAC molecules specifically bind various cellular proteins, e.g. ubiquitin proteases and ubiquitinylated proteins. In certain embodiments of a method described herein, the chimeric SURTAC molecule provided herein binds to a ubiquitinylated protein. In certain embodiments of a method described herein, the chimeric SURTAC molecule provided herein binds to a ubiquitin protease. In certain embodiments of a method described herein, the chimeric SURTAC molecule provided herein binds to both a ubiquitinylated protein and a ubiquitin protease. In certain embodiments of a method described herein, the chimeric SURTAC molecule provided herein binds to a ubiquitinylated protein, to a ubiquitin protease, or to both a ubiquitinylated protein and a ubiquitin protease. A non-limiting example of one embodiment of methods of use of the chimeric SURTAC molecules provided herein, in which the chimeric SURTAC molecule enters a cell, binds a ubiquitinylated protein and a ubiquitin protease, and releases the deubiquitinated protein, is presented in FIG. 3.

In certain embodiments of methods of use described herein, the chimeric SURTAC molecules provided herein, further comprise a third binding domain that binds to an antigen presented on a target cell. As would be appreciated by those skilled in the art, a third binding domain that specifically targets an antigen presented on a cell, or on a specific population of cells, allows delivery of the chimeric molecules provided herein to predefined cells, e.g. by binding to their cluster of differentiation (CD) molecules presented on their membrane.

In certain embodiments of methods of use of a chimeric SURTAC molecule described herein, the chimeric SURTAC molecules further comprise a cell-penetrating tag. As would be appreciated by those skilled in the art, a cell-penetrating tag that increases the entry of the chimeric SURTAC molecules into cells, allows efficient delivery of the chimeric molecules to cells. In some embodiments, cell-penetrating tags comprise cell-penetrating peptides (CPPs). CPPs in some embodiments comprise short peptides that facilitate cellular intake/uptake of various molecules. In some embodiments of methods of use of chimeric SURTAC molecules described herein, the chimeric molecules are associated with the CPPs either through chemical linkage via covalent bonds or through non-covalent interactions.

In certain embodiments of methods of use of chimeric SURTAC molecules provided herein, the method of use restores normal cell function, in cells which have been challenged by an insult. In some embodiments of methods of use of a chimeric SURTAC molecule, the method partially restores normal cell function in cells, which have been challenged by an insult. An insult to a cell may be temporary or permanent, destructive or not destructive. In some embodiments of methods of use, chimeric SURTAC molecules restore or partially restore function to cells experiencing an insult such as thermal shock, structural damage, infection and neoplastic transformation. While certain insults may be rectified by cellular machinery, other insults may be determining the fate of the cell to death, e.g. by necrosis or apoptosis. In some embodiments, methods of use described herein are beneficial to artificially manipulate the fate of the cell.

In some embodiments, in order to regain homeostasis after an insult, methods of use of a chimeric SURTAC molecule described herein may be beneficial to decrease the degradation of ubiquitinylated homeostasis proteins. In certain embodiments, the first binding domain binds to a ubiquitinylated homeostasis protein. In certain embodiments, the ubiquitinylated homeostasis protein comprises a wild-type, functional protein. In certain embodiments, the first binding domain binds to a ubiquitinylated p53 protein. In certain embodiments, the second binding domain binds to a deubiquitinating enzyme of p53. In certain embodiments, the deubiquitinating enzyme of p53 comprises a USP5, USP7, USP9X, USP10, USP11, USP24, USP29, USP42, OTUD1, OTUD5, Ataxin-3, USP28, or USP49 protease. In certain embodiments, the deubiquitinating enzyme of p53 is USP5. In certain embodiments, the deubiquitinating enzyme of p53 is USP7. In certain embodiments, the deubiquitinating enzyme of p53 is USP9X. In certain embodiments, the deubiquitinating enzyme of p53 is USP10. In certain embodiments, the deubiquitinating enzyme of p53 is USP11. In certain embodiments, the deubiquitinating enzyme of p53 is USP24. In certain embodiments, the deubiquitinating enzyme of p53 is USP29. In certain embodiments, the deubiquitinating enzyme of p53 is USP42. In certain embodiments, the deubiquitinating enzyme of p53 is OTUD1. In certain embodiments, the deubiquitinating enzyme of p53 is OTUD5. In certain embodiments, the deubiquitinating enzyme of p53 is Ataxin-3. In certain embodiments, the deubiquitinating enzyme of p53 is USP28. In certain embodiments, the deubiquitinating enzyme of p53 is USP49.

In tumors that express a wild-type, functional p53 protein, its tumor-suppressor function is often impaired by HDM2 that binds to p53 and targets p53 for proteasomal degradation. RITA ((2,5-bis(5-hydroxymethyl-2-thienyl)furan) (reactivation of p53 and induction of tumor cell apoptosis)) binds to p53 and induces its accumulation in tumor cells. RITA prevents p53-HDM-2 interaction in vitro and in vivo and affects p53 interaction with several negative regulators. RITA induced expression of p53 target genes and massive apoptosis in various tumor cells lines expressing wild-type p53 (Issaeva et al., 2004, Nature Medicine, volume 10, pages 1321-1328).

In certain embodiments, the first binding domain of a chimeric SURTAC molecule comprises a RITA. In certain embodiments of methods of use herein, the first binding domain of a chimeric SURTAC molecule binds to a RITA. RITA is a non-limiting example of a molecule, or an “intermediary molecule”, which is both specific to binding p53 and is not inhibitory to p53 cellular functions.

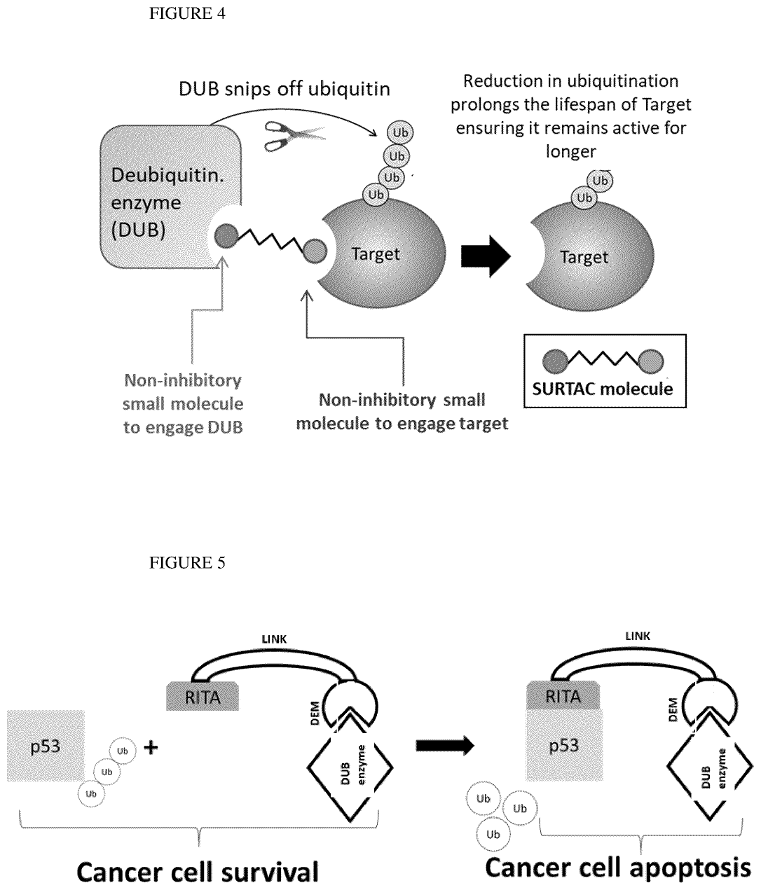

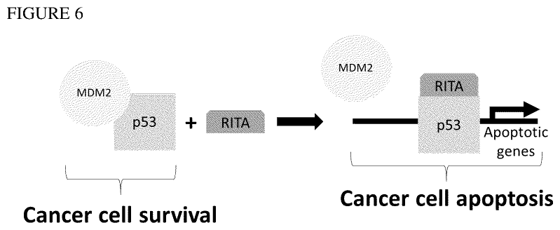

In some embodiments, use of RITA may be beneficial wherein RITA can be used to specifically target p53 proteins by the chimeric molecules provided herein. In certain embodiments, the first binding domain of the chimeric molecules provided herein comprises RITA, as illustrated in FIG. 5. In certain embodiments, RITA can be used to release p53 proteins from MDM2, thereby allowing the p53 proteins to promote an apoptotic cascade, as illustrated in FIG. 6, e.g. in combination with a method of use of the chimeric molecules provided herein.

In some embodiments of method of use of a chimeric SURTAC molecule described herein, a chimeric molecule is used in a method for preventing, reducing, or ameliorating the degradation of a ubiquitinylated protein, the method comprising contacting the ubiquitinylated protein with a chimeric SURTAC molecule comprising a first binding domain, a second binding domain and a linker, wherein: the first binding domain is configured to bind to an ubiquitinylated protein; the second binding domain is configured to bind to an ubiquitin protease that cleaves ubiquitin from the ubiquitinylated protein bound to the first binding domain, and the linker domain is configured to link the first binding domain to the second binding domain; thereby preventing, reducing, or ameliorating the degradation of the ubiquitinylated protein.

In some embodiments of method of use of a chimeric SURTAC molecule described herein, the ubiquitinylated protein is a homeostasis protein. A skilled artisan would appreciate that numerous homeostasis proteins are known in the art, for example the homeostasis proteins described in Uhlén et al., (2015) Science. January 23; 347(6220):1260419, the list of which is incorporated herein in its entirety. In some embodiments of method of use of a chimeric SURTAC molecule described herein, a homeostasis protein may be any homeostasis protein known in the art.

In some embodiments of method of use of a chimeric SURTAC molecule described herein, the ubiquitinylated protein is a tumor suppressor protein. In some embodiments of method of use of a chimeric SURTAC molecule described herein, the tumor suppressor protein is selected from the group consisting of p53, phosphatase and tensin homolog (PTEN), von Hippel-Lindau tumor suppressor (pVHL), adenomatous polyposis coli (APC), cluster of differentiation 95 (CD95), suppression of tumorigenicity 5 (ST5), Yippee-like 3 (YPEL3), and mammalian target of rapamycin (mTOR).

Tumor protein p53, also known as p53, cellular tumor antigen p53 (UniProt name), phosphoprotein p53, tumor suppressor p53, antigen NY-CO-13, or transformation-related protein 53 (TRP53), comprises any isoform of a protein encoded by homologous genes in various organisms, such as TP53 (humans) and Trp53 (mice). p53 has been described as “the guardian of the genome” because of its role in conserving stability by preventing genome mutation. Hence TP53 is classified as a tumor suppressor gene. In some embodiments of method of use of a chimeric SURTAC molecule described herein, the tumor suppressor protein is p53.

Phosphatase and tensin homolog (PTEN) is a protein that, in humans, is encoded by the PTEN gene. Mutations of this gene are a step in the development of many cancers. PTEN acts as a tumor suppressor gene through the action of its phosphatase protein product. This phosphatase is involved in the regulation of the cell cycle, preventing cells from growing and dividing too rapidly. In some embodiments of method of use of a chimeric SURTAC molecule described herein, the tumor suppressor protein is PTEN.

The von Hippel-Lindau tumor suppressor also known as pVHL is a protein that in humans is encoded by the VHL gene. Mutations of the VHL gene are associated with von Hippel-Lindau disease. In some embodiments of method of use of a chimeric SURTAC molecule described herein, the tumor suppressor protein is pVHL.

Adenomatous polyposis coli (APC) also known as deleted in polyposis 2.5 (DP2.5) is a protein that in humans is encoded by the APC gene. The APC protein is a negative regulator that controls beta-catenin concentrations and interacts with E-cadherin, which are involved in cell adhesion. Mutations in the APC gene may result in colorectal cancer. In some embodiments of method of use of a chimeric SURTAC molecule described herein, the tumor suppressor protein is APC.

Fas or FasR, also known as apoptosis antigen 1 (APO-1 or APT), cluster of differentiation 95 (CD95) or tumor necrosis factor receptor superfamily member 6 (TNFRSF6) is a protein that in humans is encoded by the FAS gene. The Fas receptor is a death receptor on the surface of cells that leads to programmed cell death (apoptosis). In some embodiments of method of use of a chimeric SURTAC molecule described herein, the tumor suppressor protein is CD95.

Suppression of tumorigenicity 5 is a protein that in humans is encoded by the ST5 gene. In some embodiments of method of use of a chimeric SURTAC molecule described herein, the tumor suppressor protein is ST5.

Yippee-like 3 is a protein that in humans is encoded by the YPEL3 gene. YPEL3 has growth inhibitory effects in normal and tumor cell lines. Induction of YPEL3 has been shown to trigger permanent growth arrest or cellular senescence in certain human normal and tumor cell types. In some embodiments of method of use of a chimeric SURTAC molecule described herein, the tumor suppressor protein is YPEL3.

The mammalian target of rapamycin (mTOR), also known as the mechanistic target of rapamycin and FK506-binding protein 12-rapamycin-associated protein 1 (FRAP1), is a kinase that in humans is encoded by the MTOR gene. In some embodiments of method of use of a chimeric SURTAC molecule described herein, the tumor suppressor protein is mTOR.

In some embodiments of method of use of a chimeric SURTAC molecule described herein, (i) the ubiquitinylated protein is p53, (ii) the second binding domain binds to a deubiquitinating enzyme selected from the group consisting of USP5, USP7, USP9X, USP10, USP11, USP24, USP29, USP42, OTUD1, OTUD5, Ataxin-3, USP28, and USP49, (iii) the first binding domain comprises or binds to a RITA small molecule, or (iv) any combination of (i), (ii) and (iii).

In some embodiments of method of use of a chimeric SURTAC molecule described herein, the ubiquitinylated protein is a neuroprotective protein. In some embodiments of method of use of a chimeric SURTAC molecule described herein, the neuroprotective protein is selected from the group consisting of ciliary neurotrophic factor (CTNF), insulin-like growth factor 1 (IGF-1), vascular endothelial growth factor (VEGF), and brain-derived neurotrophic factor (BDNF).

Ciliary neurotrophic factor is a protein that in humans is encoded by the CNTF gene. The protein encoded by this gene is a polypeptide hormone and neurotrophic factor which promotes neurotransmitter synthesis and neurite outgrowth in neural populations. In some embodiments of method of use of a chimeric SURTAC molecule described herein, the neuroprotective protein is CTNF.

Insulin-like growth factor 1 (IGF-1), also called somatomedin C, is a protein that in humans is encoded by the IGF1 gene. IGF-1 is a hormone similar in molecular structure to insulin. It plays an important role in childhood growth and continues to have anabolic effects in adults. In some embodiments of method of use of a chimeric SURTAC molecule described herein, the neuroprotective protein is IGF-1.

Vascular endothelial growth factor (VEGF), originally known as vascular permeability factor (VPF), is a signal protein produced by cells that stimulates the formation of blood vessels. VEGF is a sub-family of growth factors, the platelet-derived growth factor family of cystine-knot growth factors. They are important signaling proteins involved in both vasculogenesis (the de novo formation of the embryonic circulatory system) and angiogenesis (the growth of blood vessels from pre-existing vasculature). In some embodiments of method of use of a chimeric SURTAC molecule described herein, the neuroprotective protein is a VEGF.

Brain-derived neurotrophic factor, also known as BDNF, is a protein that, in humans, is encoded by the BDNF Gene. BDNF is a member of the neurotrophin family of growth factors, which are related to the canonical nerve growth factor. In some embodiments of method of use of a chimeric SURTAC molecule described herein, the neuroprotective protein is BDNF.

In some embodiments a method of use of a chimeric SURTAC molecule described herein comprises a method of use of a chimeric molecule for modulating the activity of a ubiquitinylated protein, the method comprising contacting the ubiquitinylated protein with a chimeric SURTAC molecule comprising a first binding domain, a second binding domain, and a linker domain, wherein:
the first binding domain is configured to bind to an ubiquitinylated protein;
the second binding domain is configured to bind to an ubiquitin protease that cleaves ubiquitin from the ubiquitinylated protein bound to the first binding domain, and
the linker domain is configured to link the first binding domain to the second binding domain;
   thereby modulating the activity of the ubiquitinylated protein.

In some embodiments, a method of use of a chimeric SURTAC molecule described herein comprises a use for modulating the cellular location of a ubiquitinylated protein, the method comprising contacting the ubiquitinylated protein with a survival-targeting chimeric (SURTAC) molecule comprising a first binding domain, a second binding domain, and a linker domain, wherein:
the first binding domain is configured to bind to an ubiquitinylated protein;
the second binding domain is configured to bind to an ubiquitin protease that cleaves ubiquitin from the ubiquitinylated protein bound to the first binding domain, and
the linker domain is configured to link the first binding domain to the second binding domain; thereby modulating the cellular location of the ubiquitinylated protein.

In some embodiments, a method of use of a chimeric SURTAC molecule described herein comprises a use for modulating the interaction of a ubiquitinylated protein with another protein, the method comprising contacting the ubiquitinylated protein with a survival-targeting chimeric (SURTAC) molecule comprising a first binding domain, a second binding domain, and a linker domain, wherein:
the first binding domain is configured to bind to an ubiquitinylated protein;
the second binding domain is configured to bind to an ubiquitin protease that cleaves ubiquitin from the ubiquitinylated protein bound to the first binding domain, and
the linker domain is configured to link the first binding domain to the second binding domain; thereby modulating the interaction of the ubiquitinylated protein with the other protein.

In some embodiments, a method of use of a chimeric SURTAC molecule described herein comprises a use for restoring homeostasis in a cell, the method comprising contacting the cell with a survival-targeting chimeric (SURTAC) molecule comprising a first binding domain, a second binding domain, and a linker domain, wherein:
the first binding domain is configured to bind to an ubiquitinylated homeostasis protein;
the second binding domain is configured to bind to an ubiquitin protease that cleaves ubiquitin from the ubiquitinylated protein bound to the first binding domain, and
the linker domain is configured to link the first binding domain to the second binding domain; thereby restoring homeostasis in the cell.

In some embodiments, a method of use of a chimeric SURTAC molecule described herein comprises a use for treating, reducing, or ameliorating cancer in a subject, the method comprising administering to the subject a survival-targeting chimeric (SURTAC) molecule comprising a first binding domain, a second binding domain, and a linker domain, wherein:
the first binding domain is configured to bind to an ubiquitinylated tumor suppressor protein;
the second binding domain is configured to bind to an ubiquitin protease that cleaves ubiquitin from the ubiquitinylated protein bound to the first binding domain, and
the linker domain is configured to link the first binding domain to the second binding domain; thereby treating, reducing, or ameliorating cancer in the subject.

In certain embodiments of methods of use for treating, reducing, or ameliorating cancer in a subject, the ubiquitinylated tumor suppressor protein comprises a wild-type, functional tumor suppressor protein. In certain embodiments of methods of use for treating, reducing, or ameliorating cancer in a subject, the tumor suppressor protein is selected from the group consisting of p53, PTEN, pVHL, APC, CD95, ST5, YPEL3, and mTor. In certain embodiments of methods of use for treating, reducing, or ameliorating cancer in a subject, the tumor suppressor protein is p53. In certain embodiments of methods of use for treating, reducing, or ameliorating cancer in a subject, the tumor suppressor protein is PTEN. In certain embodiments of methods of use for treating, reducing, or ameliorating cancer in a subject, the tumor suppressor protein is pVHL. In certain embodiments of methods of use for treating, reducing, or ameliorating cancer in a subject, the tumor suppressor protein is APC. In certain embodiments of methods of use for treating, reducing, or ameliorating cancer in a subject, the tumor suppressor protein CD95. In certain embodiments of methods of use for treating, reducing, or ameliorating cancer in a subject, the tumor suppressor protein is ST5. In certain embodiments of methods of use for treating, reducing, or ameliorating cancer in a subject, the tumor suppressor protein is YPEL3. In certain embodiments of methods of use for treating, reducing, or ameliorating cancer in a subject, the tumor suppressor protein is mTor. In certain embodiments of methods of use for treating, reducing, or ameliorating cancer in a subject, the ubiquitinylated tumor suppressor protein is p53, the second binding site binds to a deubiquitinating enzyme selected from the group consisting of USP5, USP7, USP9X, USP10, USP11, USP24, USP29, USP42, OTUD1, OTUD5, Ataxin-3, USP28, and USP49, the first binding site comprises or binds to a RITA small molecule, or (iv) any combination thereof.

In some embodiments, a method of use of a chimeric SURTAC molecule described herein comprises a use for treating, reducing, or ameliorating neuronal damage in a subject, the method comprising administering to the subject a survival-targeting chimeric (SURTAC) molecule comprising a first binding domain, a second binding domain, and a linker domain, wherein:
the first binding domain is configured to bind to an ubiquitinylated neuroprotective protein;
the second binding domain is configured to bind to an ubiquitin protease that cleaves ubiquitin from the ubiquitinylated protein bound to the first binding domain, and
the linker domain is configured to link the first binding domain to the second binding domain; thereby treating, reducing, or ameliorating neuronal damage in the subject.

In certain embodiments of the method of use of a chimeric SURTAC molecule for treating, reducing, or ameliorating neuronal damage in a subject, the ubiquitinylated neuroprotective protein comprises a wild-type, functional neuroprotective protein. In certain embodiment of the method of use of a chimeric SURTAC molecule for treating, reducing, or ameliorating neuronal damage in a subject, the neuroprotective protein is selected from the group consisting of CTNF, IGF-1, VEGF, and BDNF. In certain embodiment, the neuroprotective protein is CTNF. In certain embodiment of the method of use of a chimeric SURTAC molecule for treating, reducing, or ameliorating neuronal damage in a subject, the neuroprotective protein is IGF-1. In certain embodiment of the method of use of a chimeric SURTAC molecule for treating, reducing, or ameliorating neuronal damage in a subject, the neuroprotective protein is VEGF. In certain embodiment of the method of use of a chimeric SURTAC molecule for treating, reducing, or ameliorating neuronal damage in a subject, the neuroprotective protein is BDNF.

### Definitions

It should be appreciated that the chimeric molecules provided herein are alternatively described as “synthetic”, “multi-domain”, “double domain”, “triple-domain”, “multi-functional”, “bi-functional”, “bi-specific”, “tri-specific”, and/or “chimeric” to characterize different attributes of the chimeric molecules provided herein, and therefore each term may be used alone or in combination with any other term to define the chimeric molecules.

The term “synthetic molecule” generally means that the referenced molecule is man-made and is not found in nature.

The term “multi-domain molecule” generally means that the referenced molecule comprises at least two different structural domains. The chimeric molecules provided herein have two different mandatory domains—a first binding domain in charge of binding ubiquitinylated proteins, and a second binding domain in charge of binding DUB enzymes. The term “double-domain molecule” generally means that the referenced molecule comprises two functional domains, i.e. the first and second binding domains as described above. The term “triple-domain molecule” generally means that the referenced molecule comprises three functional domains, i.e. the first and second binding domains as described above, and another domain, e.g. comprising a binding domain that binds to an antigen presented on a target cell and/or a cell-penetrating tag.

The term “multi-functional molecule” generally means that the referenced molecule can perform a plurality of functions. The chimeric molecules provided herein have two different mandatory functions—binding DUB enzymes and binding ubiquitinylated proteins, and as such may be also referred to as “bi-functional”. The term “tri-functional molecule” generally means that the referenced molecule has the two mandatory functions, and another function, e.g. binding to an antigen presented on a target cell and/or penetrating a cell.

The term “chimeric molecule” generally means that the referenced molecule is made of two or more different domains or structures that are not found together in nature in a single molecule. The chimeric molecules provided herein are considered chimeric as they comprise at least two different domains or structures that are not found together in nature in a single molecule, i.e. the first and second binding domains as described above. In nature, no molecule has been found to specifically and simultaneously bind DUB enzymes and ubiquitinylated proteins as described herein.

It will be understood by those skilled in the art that the term “ubiquitin protease” or “deubiquitinating enzyme” generally refers to a protein which is a protease enzyme that is capable of cleaving one or more ubiquitin molecules from proteins and other molecules. DUBs can cleave the peptide or iso-peptide bond between ubiquitin and its substrate protein. In one embodiment, DUBs can cleave the bond between an ubiquitin molecule and a substrate protein and/or the bond between an ubiquitin molecule and an adjacent ubiquitin molecule on the same ubiquitin chain.

It will be understood by those skilled in the art that the term “binding domain”, as in “first binding domain” and “second binding domain”, generally refers to a part of a molecule which specifically targets, or is specifically recognized by, a separate molecule. The first binding domain may specifically target, or be specifically recognized by, an ubiquitinylated protein, i.e. the protein of interest from which one or more ubiquitin molecules would be ultimately removed. The second binding domain may specifically target, or be specifically recognized by, a deubiquitinating enzyme, i.e. a protease that cleaves ubiquitin from proteins and other molecules, i.e. the enzyme which would ultimately remove one or more ubiquitin molecules from the protein of interest.

It will be understood by those skilled in the art that the term “linker” or “linking domain” generally refers to a part of a molecule which links, connects, associates or otherwise interacts with a plurality of other molecules. In one embodiment, the linker domain of the chimeric molecules provided herein connects or links the first binding domain to the second binding domain of the chimeric molecules provided herein.

The term “antibody” or “antibodies” is used herein in a broad sense and includes both polyclonal and monoclonal antibodies. In addition to intact or “full” immunoglobulin molecules, also included in the term “antibodies” are fragments (e.g. CDRs, Fv, Fab and Fc fragments) or polymers of those immunoglobulin molecules and humanized versions of immunoglobulin molecules. The antibodies may also be generated using well-known methods. In one embodiment, the second binding domain of the chimeric molecules provided herein may comprise an antibody that binds DUB or an ubiquitin-protease-binding fragment thereof, and the first binding domain of the chimeric molecules provided herein may be an antibody that binds ubiquitinylated protein or an ubiquitinylated-protein-binding fragment thereof.

The term “antibody” as used herein further includes Fab, the fragment which contains a monovalent antigen-binding fragment of an antibody molecule, which can be produced by digestion of whole antibody with the enzyme papain to yield an intact light chain and a portion of one heavy chain; Fab′, the fragment of an antibody molecule that can be obtained by treating whole antibody with pepsin, followed by reduction, to yield an intact light chain and a portion of the heavy chain, two Fab′ fragments are obtained per antibody molecule; (Fab′)2, the fragment of the antibody that can be obtained by treating whole antibody with the enzyme pepsin without subsequent reduction, F(ab′)2 is a dimer of two Fab′ fragments held together by two disulfide bonds; Fv, a genetically engineered fragment containing the variable region of the light chain and the variable region of the heavy chain expressed as two chains; and single chain antibody (“SCA”), a genetically engineered molecule containing the variable region of the light chain and the variable region of the heavy chain, linked by a suitable polypeptide linker as a genetically fused single chain molecule.

In one embodiment, Fv fragments comprise an association of VH and VL chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Alternatively, the Fv fragments comprise VH and VL chains connected by a peptide linker.

The term “antibody” as used herein further includes a peptide coding for one or more complementarity-determining regions (CDRs). In one embodiment, CDR peptides (“minimal recognition units”) can be obtained by constructing genes encoding the CDR of an antibody of interest.

As used herein the term “peptide” includes native peptides (either degradation products, synthetically synthesized peptides or recombinant peptides) and peptidomimetics (typically, synthetically synthesized peptides), such as peptoids and semipeptoids which are peptide analogs, which may have, for example, modifications rendering the peptides more stable while in a body or more capable of penetrating into bacterial cells. Such modifications include, but are not limited to N terminus modification, C terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modifications, and residue modification.

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH₂—), α-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom. These modifications can occur at any of the bonds along the peptide chain and even at several (e.g. 2-3) at the same time. Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr. In addition to the above, the linkers of the chimeric molecules provided herein may also include one or more modified amino acids or one or more non-amino acid monomers (e.g. fatty acids, complex carbohydrates, etc.).

As used herein the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

It will be understood by those skilled in the art that the term “ligand” generally refers to a substance, such as a small molecule, that forms a complex with another biomolecule. In one embodiment, the first binding domain of the chimeric molecules provided herein may comprise a ligand that binds an ubiquitinylated protein, and the second binding domain of the chimeric molecules provided herein may comprise a ligand that binds an ubiquitin protease.

It will be understood by those skilled in the art that the term “ubiquitinylated protein” generally refers to the protein of interest, from which one or more ubiquitin molecules would be ultimately removed. As would be appreciated by those skilled in the art, an “ubiquitinylated protein” may carry a single ubiquitin molecule, multiple ubiquitin molecules, a single ubiquitin chain, multiple ubiquitin chains, linear ubiquitin chains, branched ubiquitin chains, or any combination thereof. In one embodiment, the first binding domain of the chimeric molecules provided herein could bind to an ubiquitinylated protein, i.e. a protein covalently attached to at least one ubiquitin molecule.

It will be understood by those skilled in the art that the terms “catalytic domain” and “active site” are interchangeable, and generally refer to the region of an enzyme where substrate molecules bind and undergo a chemical reaction. For example, the catalytic domain of cysteine protease deubiquitinating enzymes (DUBs) use either catalytic diads or triads (either two or three amino acids) to catalyse the hydrolysis of the amide bonds between ubiquitin and the substrate. The active site residues that contribute to the catalytic activity of the cysteine protease DUBs are cysteine (diad/triad), histidine (diad/triad) and aspartate or asparagine (triad only). The histidine is polarised by the aspartate or asparagine in catalytic triads or by other ways in diads. This polarised residue lowers the pKa of the cysteine, allowing it to perform a nucleophilic attack on the isopeptide bond between the ubiquitin C-terminus and the substrate lysine. Metalloproteases coordinate zinc ions with histidine, aspartate and serine residues, which activate water molecules and allows them to attack the isopeptide bond.

It will be understood by those skilled in the art that the term “accessory domain” generally refers to the region of an enzyme which assist the catalytic domain of the enzyme to perform its function, e.g. by binding substrate molecules and/or participating in a chemical reaction.

It should be understood that the term “modulating” as used herein generally refers to any change of an attribute. For example, “modulating the activity of an ubiquitinylated protein” may mean increasing or decreasing the activity of an ubiquitinylated protein, “modulating the cellular location of an ubiquitinylated protein” means changing the location of an ubiquitinylated protein within a cell, and “modulating the interaction of an ubiquitinylated protein with another protein” may mean increasing or decreasing protein-protein interaction between an ubiquitinylated protein to a different protein.

It will be understood by those skilled in the art that the phrase “preventing, reducing, or ameliorating protein degradation” refers to complete stop of protein degradation, decrease in the number of proteins degraded per a time unit, or decrease in the rate in which a protein is degraded.

It will be understood by those skilled in the art that the phrase “treating, reducing, or ameliorating a disease” refers to preventing symptoms associated with a disease, decreasing symptoms associated with a disease, postponing symptoms associated with a disease, lessening the severity of a disease or curing the disease.

### EXAMPLES

### Example 1

### Cell Penetration and Targeted Deubiquitylation in Cancer Cells In-Vitro

Chimeric molecules, as disclosed herein in detail, are produced, comprising a first binding domain (a TAR engagement motif (TEM)), which specifically binds to ubiquitinylated p53 proteins, and a second binding domain (a DUB engagement motif (DEM)), which specifically binds to USP11, a known deubiquitinating protease of ubiquitinylated p53 proteins. The chimeric molecules are added to a solution of viable cancer cells, which over-ubiquitinylate and thus over-degrade p53 proteins. The solution is than mixed and incubated at 37° C. for several hours. The level of ubiquitinylation of p53 proteins in the cells, and the viability of the cells, are monitored during incubation. It is shown that the viability of the cells decreases as the level of ubiquitinylation of p53 proteins in the cells decreases.

### Example 2

### Cancer Therapy in a Murine Model of Solid Tumors

Chimeric molecules, as disclosed herein in detail, are produced, comprising a first binding domain (a TAR engagement motif), which specifically binds to ubiquitinylated p53 proteins, and a second binding domain (a DUB engagement motif), which specifically binds to USP11, a known deubiquitinating protease of ubiquitinylated p53 proteins. The chimeric molecules are intratumorally injected to nude mice carrying established solid tumors, which over-ubiquitinylate and thus over-degrade p53 proteins. A control group of mice carrying tumors is mock treated with PBS. The volume of the solid tumors, and the viability of the mice, are monitored over several weeks. It is shown that the volume of the tumors decreases as the viability of the mice increases in the chimeric-molecule-treated group. In the mock-treated group, tumors progress and quality-of-life deteriorates until mice are humanely sacrificed.

### Example 3

### Cancer Therapy in a Murine Model of Blood Cancer

Chimeric molecules, as disclosed herein in detail, are produced, comprising a first binding domain (a TAR engagement motif), which specifically binds to ubiquitinylated p53 proteins, and a second binding domain (a DUB engagement motif), which specifically binds to USP11, a known deubiquitinating protease of ubiquitinylated p53 proteins. The chimeric molecules are systemically injected to nude mice afflicted with lymphoma, which over-ubiquitinylate and thus over-degrade p53 proteins. A control group of mice afflicted with lymphoma is mock treated with PBS. The viability of the mice is monitored during several weeks. It is shown that the viability of the mice increases in the chimeric-molecule-treated group. In the mock-treated group, quality-of-life deteriorates until mice are humanely sacrificed.

### Example 4

### Cell Penetration and Targeted Deubiquitylation in ΔF508 CFTR Cells In-Vitro

Chimeric molecules, as disclosed herein in detail, are produced, comprising a first binding domain (a TAR engagement motif), which specifically binds to ubiquitinylated ΔF508 cystic fibrosis transmembrane conductance regulator (CFTR) proteins, and a second binding domain (a DUB engagement motif), which specifically binds to a known deubiquitinating protease of ubiquitinylated CFTR proteins. The chimeric molecules are added to a solution of primary viable ΔF508 CFTR cells, which ubiquitinylate and thus degrade ΔF508 CFTR proteins. The solution is than mixed and incubated at 37° C. for several hours. The level of ubiquitinylation of ΔF508 CFTR proteins in the cells, and the viability of the cells, are monitored during incubation. It is shown that the viability of the cells increases as the level of ubiquitinylation of ΔF508 CFTR proteins in the cells decreases.

### Example 5

### Cell Penetration and Targeted Deubiquitylation in Papilloma-Infected Cells In-Vitro

Chimeric molecules, as disclosed herein in detail, are produced, comprising a first binding domain (a TAR engagement motif), which specifically binds to ubiquitinylated p53 proteins, and a second binding domain (a DUB engagement motif), which specifically binds to USP11, a known deubiquitinating protease of ubiquitinylated p53 proteins. The chimeric molecules are added to a solution of viable epithelial cells infected by human papillomavirus 16 (HPV16), which over-ubiquitinylates and thus over-degrades p53 proteins. The solution is then mixed and incubated at 37° C. for several hours. The level of ubiquitinylation of p53 proteins in the cells, and the viability of the cells, are monitored during incubation. It is shown that the viability of the cells decreases as the level of ubiquitinylation of p53 proteins in the cells decreases.

What is claimed is:

1. A survival-targeting chimeric (SURTAC) molecule comprising a first binding domain, a second binding domain, and a linker domain, wherein:
(i) the first binding domain is configured to bind to an ubiquitinylated protein, wherein the ubiquitinylated protein carries one or more ubiquitin molecules;
(ii) the second binding domain is configured to bind to an ubiquitin protease that cleaves ubiquitin from the ubiquitinylated protein bound to the first binding domain, wherein said ubiquitin protease cleaves one or more ubiquitin molecules from said ubiquitinylated protein; and
(iii) the linker domain is configured to link the first binding domain to the second binding domain.

2. The chimeric molecule of claim 1, wherein the first binding domain comprises a peptide or a small molecule.

3. The chimeric molecule of claim 1, wherein the first binding domain is configured to directly bind to the ubiquitinylated protein.

4. The chimeric molecule of claim 3, wherein the first binding domain comprises
(a) an antibody or an antigen-binding fragment thereof that binds to the ubiquitinylated protein; or
(b) a ligand that binds to the ubiquitinylated protein.

5. The chimeric molecule of claim 1, wherein the first binding domain binds an intermediate molecule that binds to the ubiquitinylated protein.

6. The chimeric molecule of claim 5, wherein the intermediate molecule comprises
(a) an antibody or an antigen-binding fragment thereof that binds to the ubiquitinylated protein; or
(b) a ligand that binds to the ubiquitinylated protein.

7. The chimeric molecule of claim 1, wherein the first binding domain transiently binds to the ubiquitinylated protein and dissociates from said protein after one or more ubiquitin molecules are cleaved from said ubiquitinylated protein.

8. (canceled)

9. (canceled)

10. The chimeric molecule of claim 1, wherein the ubiquitinylated protein interacts with ubiquitin protease USP5, or ubiquitin protease USP7, or ubiquitin protease USP10.

11. The chimeric molecule of claim 10, wherein when the ubiquitin protease is USP5, the ubiquitinylated protein comprises CACNA1H (Voltage-dependent T-type calcium channel subunit alpha-1H), FOXM1 (Forkhead box protein M1), MAF (Transcription factor Maf), SMURF1 (E3 ubiquitin-protein ligase SMURF1), or TRIML1 (Tripartite motif family-like protein 1).

12. (canceled)

13. The chimeric molecule of claim 10, wherein when the ubiquitin protease is USP7, the ubiquitinylated protein comprises UVSSA (UV-stimulated scaffold protein A), XPC (Xeroderma pigmentosum group C-complementing protein), ABL1 (Abelson tyrosine-protein kinase 1), AR (Androgen receptor), ATXN1 (Ataxin-1), CHEK1 (Serine/threonine-protein kinase Chk1), CHFR (E3 ubiquitin-protein ligase CHFR), CLSPN (Claspin), CSNK2A1 (Casein kinase II subunit alpha), DAXX (Death domain-associated protein 6), DNMT1 (DNA (cytosine-5)-methyltransferase 1), FOXO1 (Forkhead box protein O1), FOXO4 (Forkhead box protein O4), GMPS (GMP synthetase), IFNAR1 (Type I interferon receptor 1), IKBKG (I-kappa-B kinase subunit gamma), KAT5 (Histone acetyltransferase KAT5), KDM1A (Lysine-specific histone demethylase 1A), MARCHF7 (Membrane Associated Ring-CH-Type Finger 7), MDM2 (E3 ubiquitin-protein ligase Mdm2), MDM4 (Mdm2-like p53-binding protein), MEX3C (RNA-binding E3 ubiquitin-protein ligase MEX3C), MYC (Myc proto-oncogene protein), MYD88 (Myeloid differentiation primary response protein MyD88), PML (Promyelocytic leukemia protein), POLH (DNA polymerase theta), PPARG (Peroxisome proliferator-activated receptor gamma), PTEN (Phosphatidylinositol 3,4,5-trisphosphate 3-phosphatase and dual-specificity protein phosphatase), RAD18 (E3 ubiquitin-protein ligase RAD18), RARA (Retinoic acid receptor alpha), RB1 (Retinoblastoma-associated protein), RELA (Transcription factor p65), RNF168 (E3 ubiquitin-protein ligase RNF168), RNF220 (E3 ubiquitin-protein ligase RNF220), SKP1 (S-phase kinase-associated protein 1), TP53 (Cellular tumor antigen p53), TRAF6 (TNF receptor-associated factor 6), TRIP12 (E3 ubiquitin-protein ligase TRIP12), or TRRAP (Transformation/transcription domain-associated protein).

14. (canceled)

15. The chimeric molecule of claim 10, wherein when the ubiquitin protease is USP10, the ubiquitinylated protein comprises AR (Androgen receptor), ATM (Serine-protein kinase ATM), CFTR (Cystic fibrosis transmembrane conductance regulator), EIF4G1 (Eukaryotic translation initiation factor 4 gamma 1), MSH2 (DNA mismatch repair protein Msh2), PRKAA1 (5′-AMP-activated protein kinase catalytic subunit alpha-1), PTEN (Phosphatase and tensin homolog), or TBX21 (T-box transcription factor TBX21).

16. The chimeric molecule of claim 1, wherein the ubiquitinylated protein is a non-natural target of ubiquitin protease.

17. The chimeric molecule of claim 1, wherein the second binding domain comprises a peptide or a small molecule.

18. The chimeric molecule of claim 1, wherein the second binding domain is configured to directly bind to the ubiquitin protease.

19. The chimeric molecule of claim 18 wherein the second binding domain comprises
(a) an antibody or an antigen-binding fragment thereof that binds to the ubiquitin protease; or
(b) a ligand that binds to the ubiquitin protease.

20. The chimeric molecule of claim 1, wherein the second binding domain binds an intermediate molecule that binds to the ubiquitin protease.

21. The chimeric molecule of claim 20, wherein the intermediate molecule comprises
(a) an antibody or an antigen-binding fragment thereof that binds to the ubiquitin protease; or
(b) a ligand that binds to the ubiquitin protease.

22. The chimeric molecule of claim 1, wherein the ubiquitin protease comprises a domain selected from the group consisting of ubiquitin-specific proteases (DUSP) domain, ubiquitin-like (UBL) domain, meprin and TRAF homology (MATH) domain, zinc-finger ubiquitin-specific protease (ZnF-UBP) domain, zinc-finger myeloid, nervy and DEAF1 (ZnF-MYND) domain, ubiquitin-associated (UBA) domain, CHORD-SGT1 (CS) domain, microtubule-interacting and trafficking (MIT) domain, rhodenase-like domain, TBC/RABGAP domain, and B-box domain, and any combination thereof.

23. The chimeric molecule of claim 1, wherein the ubiquitin protease is from a family selected from the group consisting of ubiquitin specific proteases (USP) family, ovarian tumor proteases (OUT) family, ubiquitin C-terminal hydrolases (UCH) family, Josephin domain family (Josephin), motif interacting with ubiquitin-containing novel deubiquitinase family (MINDY), and JAB1/MPN/Mov34 metalloenzyme domain family (JAMM).

24. (canceled)

25. The chimeric molecule of claim 1, wherein the linker domain comprises a peptide or a small molecule.

26. The chimeric molecule of claim 1, wherein the linker domain covalently links the first binding domain to the second binding domain.

27. The chimeric molecule of claim 1, wherein the linker domain non-covalently links the first binding domain to the second binding domain.

28. The chimeric molecule of claim 1, wherein the linker domain comprises
(a) a structure selected from the group consisting of polyethylene glycol, an aromatic group, an alkyl, an alkenyl, an alkyl phosphate, an alkyl siloxane, an epoxy, an acylhalide, a glycidyl, a carboxylate, and an anhydride; or
(b) a polypeptide of natural or synthetic source having a chain length of between 2 to 18 carbon atoms.

29. A method for removing at least one ubiquitin molecule from a ubiquitinylated protein, the method comprising contacting the ubiquitinylated protein with a survival-targeting chimeric (SURTAC) molecule comprising a first binding domain, a second binding domain, and a linker domain, wherein:
(i) the first binding domain is configured to bind to an ubiquitinylated protein;
(ii) the second binding domain is configured to bind to an ubiquitin protease that cleaves ubiquitin from the ubiquitinylated protein bound to the first binding domain, and
(iii) the linker domain is configured to link the first binding domain to the second binding domain;
thereby removing at least one ubiquitin molecule from a ubiquitinylated protein.

30. A method for preventing or reducing the degradation of a ubiquitinylated protein, the method comprising contacting the ubiquitinylated protein with a survival-targeting chimeric (SURTAC) molecule comprising a first binding domain, a second binding domain, and a linker domain, wherein:
(i) the first binding domain is configured to bind to an ubiquitinylated protein;
(ii) the second binding domain is configured to bind to an ubiquitin protease that cleaves ubiquitin from the ubiquitinylated protein bound to the first binding domain, and
(iii) the linker domain is configured to link the first binding domain to the second binding domain;
thereby preventing, reducing, or ameliorating the degradation of the ubiquitinylated protein.

31. The method of claim 29, wherein:
(a) the ubiquitinylated protein comprises CACNA1H (Voltage-dependent T-type calcium channel subunit alpha-1H), FOXM1 (Forkhead box protein M1), MAF (Transcription factor Maf), SMURF1 (E3 ubiquitin-protein ligase SMURF1), or TRIML1 (Tripartite motif family-like protein 1), and the ubiquitin protease comprises USP5; or
(b) the ubiquitinylated protein comprises UVSSA (UV-stimulated scaffold protein A), XPC (Xeroderma pigmentosum group C-complementing protein), ABL1 (Abelson tyrosine-protein kinase 1), AR (Androgen receptor), ATXN1 (Ataxin-1), CHEK1 (Serine/threonine-protein kinase Chk1), CHFR (E3 ubiquitin-protein ligase CHFR), CLSPN (Claspin), CSNK2A1 (Casein kinase II subunit alpha), DAXX (Death domain-associated protein 6), DNMT1 (DNA (cytosine-5)-methyltransferase 1), FOXO1 (Forkhead box protein O1), FOXO4 (Forkhead box protein O4), GMPS (GMP synthetase), IFNAR1 (Type I interferon receptor 1), IKBKG (I-kappa-B kinase subunit gamma), KAT5 (Histone acetyltransferase KAT5), KDM1A (Lysine-specific histone demethylase 1A), MARCHF7 (Membrane Associated Ring-CH-Type Finger 7), MDM2 (E3 ubiquitin-protein ligase Mdm2), MDM4 (Mdm2-like p53-binding protein), MEX3C (RNA-binding E3 ubiquitin-protein ligase MEX3C), MYC (Myc proto-oncogene protein), MYD88 (Myeloid differentiation primary response protein MyD88), PML (Promyelocytic leukemia protein), POLH (DNA polymerase theta), PPARG (Peroxisome proliferator-activated receptor gamma), PTEN (Phosphatidylinositol 3,4,5-trisphosphate 3-phosphatase and dual-specificity protein phosphatase), RAD18 (E3 ubiquitin-protein ligase RAD18), RARA (Retinoic acid receptor alpha), RB1 (Retinoblastoma-associated protein), RELA (Transcription factor p65), RNF168 (E3 ubiquitin-protein ligase RNF168), RNF220 (E3 ubiquitin-protein ligase RNF220), SKP1 (S-phase kinase-associated protein 1), TP53 (Cellular tumor antigen p53), TRAF6 (TNF receptor-associated factor 6), TRIP12 (E3 ubiquitin-protein ligase TRIP12), or TRRAP (Transformation/transcription domain-associated protein), and the ubiquitin protease comprises USP7; or
(c) the ubiquitinylated protein comprises AR (Androgen receptor), ATM (Serine-protein kinase ATM), CFTR (Cystic fibrosis transmembrane conductance regulator), EIF4G1 (Eukaryotic translation initiation factor 4 gamma 1), MSH2 (DNA mismatch repair protein Msh2), PRKAA1 (5′-AMP-activated protein kinase catalytic subunit alpha-1), PTEN (Phosphatase and tensin homolog), or TBX21 (T-box transcription factor TBX21), and the ubiquitin protease comprises USP10; or
(d) wherein the ubiquitinylated protein comprises a non-natural target of the ubiquitin protease.

32. (canceled)

33. The method of claim 29, wherein the ubiquitin protease comprises
(a) a domain selected from the group consisting of ubiquitin-specific proteases (DUSP) domain, ubiquitin-like (UBL) domain, meprin and TRAF homology (MATH) domain, zinc-finger ubiquitin-specific protease (ZnF-UBP) domain, zinc-finger myeloid, nervy and DEAF1 (ZnF-MYND) domain, ubiquitin-associated (UBA) domain, CHORD-SGT1 (CS) domain, microtubule-interacting and trafficking (MIT) domain, rhodenase-like domain, TBC/RABGAP domain, and B-box domain, and any combination thereof; or
(b) is from a family selected from the group consisting of ubiquitin specific proteases (USP) family, ovarian tumor proteases (OUT) family, ubiquitin C-terminal hydrolases (UCH) family, Josephin domain family (Josephin), motif interacting with ubiquitin-containing novel deubiquitinase family (MINDY), and JAB1/MPN/Mov34 metalloenzyme domain family (JAMM); or
(c) a combination thereof.

34. The method of claim 30, wherein:
(a) the ubiquitinylated protein comprises CACNA1H (Voltage-dependent T-type calcium channel subunit alpha-1H), FOXM1 (Forkhead box protein M1), MAF (Transcription factor Maf), SMURF1 (E3 ubiquitin-protein ligase SMURF1), or TRIML1 (Tripartite motif family-like protein 1), and the ubiquitin protease comprises USP5; or
(b) the ubiquitinylated protein comprises UVSSA (UV-stimulated scaffold protein A), XPC (Xeroderma pigmentosum group C-complementing protein), ABL1 (Abelson tyrosine-protein kinase 1), AR (Androgen receptor), ATXN1 (Ataxin-1), CHEK1 (Serine/threonine-protein kinase Chk1), CHFR (E3 ubiquitin-protein ligase CHFR), CLSPN (Claspin), CSNK2A1 (Casein kinase II subunit alpha), DAXX (Death domain-associated protein 6), DNMT1 (DNA (cytosine-5)-methyltransferase 1), FOXO1 (Forkhead box protein O1), FOXO4 (Forkhead box protein O4), GMPS (GMP synthetase), IFNAR1 (Type I interferon receptor 1), IKBKG (I-kappa-B kinase subunit gamma), KAT5 (Histone acetyltransferase KAT5), KDM1A (Lysine-specific histone demethylase 1A), MARCHF7 (Membrane Associated Ring-CH-Type Finger 7), MDM2 (E3 ubiquitin-protein ligase Mdm2), MDM4 (Mdm2-like p53-binding protein), MEX3C (RNA-binding E3 ubiquitin-protein ligase MEX3C), MYC (Myc proto-oncogene protein), MYD88 (Myeloid differentiation primary response protein MyD88), PML (Promyelocytic leukemia protein), POLH (DNA polymerase theta), PPARG (Peroxisome proliferator-activated receptor gamma), PTEN (Phosphatidylinositol 3,4,5-trisphosphate 3-phosphatase and dual-specificity protein phosphatase), RAD18 (E3 ubiquitin-protein ligase RAD18), RARA (Retinoic acid receptor alpha), RB1 (Retinoblastoma-associated protein), RELA (Transcription factor p65), RNF168 (E3 ubiquitin-protein ligase RNF168), RNF220 (E3 ubiquitin-protein ligase RNF220), SKP1 (S-phase kinase-associated protein 1), TP53 (Cellular tumor antigen p53), TRAF6 (TNF receptor-associated factor 6), TRIP12 (E3 ubiquitin-protein ligase TRIP12), or TRRAP (Transformation/transcription domain-associated protein), and the ubiquitin protease comprises USP7; or
(c) the ubiquitinylated protein comprises AR (Androgen receptor), ATM (Serine-protein kinase ATM), CFTR (Cystic fibrosis transmembrane conductance regulator), EIF4G1 (Eukaryotic translation initiation factor 4 gamma 1), MSH2 (DNA mismatch repair protein Msh2), PRKAA1 (5′-AMP-activated protein kinase catalytic subunit alpha-1), PTEN (Phosphatase and tensin homolog), or TBX21 (T-box transcription factor TBX21), and the ubiquitin protease comprises USP10; or
(d) wherein the ubiquitinylated protein comprises a non-natural target of the ubiquitin protease.

35. The method of claim 30, wherein the ubiquitin protease comprises
(a) a domain selected from the group consisting of ubiquitin-specific proteases (DUSP) domain, ubiquitin-like (UBL) domain, meprin and TRAF homology (MATH) domain, zinc-finger ubiquitin-specific protease (ZnF-UBP) domain, zinc-finger myeloid, nervy and DEAF1 (ZnF-MYND) domain, ubiquitin-associated (UBA) domain, CHORD-SGT1 (CS) domain, microtubule-interacting and trafficking (MIT) domain, rhodenase-like domain, TBC/RABGAP domain, and B-box domain, and any combination thereof; or
(b) is from a family selected from the group consisting of ubiquitin specific proteases (USP) family, ovarian tumor proteases (OUT) family, ubiquitin C-terminal hydrolases (UCH) family, Josephin domain family (Josephin), motif interacting with ubiquitin-containing novel deubiquitinase family (MINDY), and JAB1/MPN/Mov34 metalloenzyme domain family (JAMM); or
(c) a combination thereof.
